(19) United States
(12) Patent Application Publication
(10) Pub. No.: US 2024/0210421 A1
(43) Pub. Date: Jun. 27, 2024

(54) Methods of Predicting Atopic Dermatitis

(71) Applicants: National Jewish Health, Denver, CO (US); Samsung Life Public Welfare Foundation, Seoul (KR)

(72) Inventors: Evgeny Berdyshev, Denver, CO (US); Elena Goleva, Denver, CO (US); Donald Y.M. Leung, Denver, CO (US); Byung Eui Kim, Greenwood Village, CO (US); Kangmo Ahn, Seoul (KR); Jihyun Kim, Seoul (KR)

(21) Appl. No.: 18/393,301

(22) Filed: Dec. 21, 2023

Related U.S. Application Data
(60) Provisional application No. 63/435,122, filed on Dec. 23, 2022.

(51) Int. Cl. G01N 33/68; G01N 33/92

(52) U.S. Cl. CPC G01N 33/6881; G01N 33/6863; G01N 33/92; G01N 2333/7155; G01N 2800/202

(57) ABSTRACT

Disclosed herein are methods of identifying and/or screening infants at risk of developing atopic dermatitis by determining the expression level of various cytokines and lipids using a skin tape stripping method.

### CROSS-REFERENCE TO RELATED APPLICATIONS

This application claims the benefit of priority under 35 U.S.C. § 119(e) to U.S. Provisional Patent Application No. 63/435,122, filed Dec. 23, 2022. The entire disclosure of U.S. Provisional Patent Application No. 63/435,122 is incorporated herein by reference.

### BACKGROUND

Atopic dermatitis (AD) is the most common inflammatory skin disease worldwide affecting nearly 30% of children (Park Y M, et al. Risk factors of atopic dermatitis in Korean schoolchildren: 2010 international study of asthma and allergies in childhood. Asian Pac J Allergy Immunol 2016; 34(1): 65-72; Abuabara K, et al. The prevalence of atopic dermatitis beyond childhood: A systematic review and meta-analysis of longitudinal studies. Allergy 2018; 73(3): 696-704). AD patients and their families suffer from significant disturbances in the quality of life and psychosocial disorders due to sleep deprivation, anxiety, and depression (Kim J., et al. Pathophysiology of atopic dermatitis: Clinical implications. Allergy Asthma Proc 2019; 40(2): 84-92). These are often accompanied by the so-called atopic march which involves the development of food allergy, allergic rhinitis, and asthma following AD (Paller A S, et al. The atopic march and atopic multimorbidity: Many trajectories, many pathways. J Allergy Clin Immunol 2019; 143(1): 46-55). This constellation of atopic co-morbidities contributes to billions of dollars in health care burden to society.

Primary or secondary epidermal barrier impairment along with immune dysregulation and skin microbial dysbiosis plays a central role in driving AD (Leung D Y M, et al. Cutaneous barrier dysfunction in allergic diseases. J Allergy Clin Immunol 2020; 145(6): 1485-97; Luger T, et al. Atopic dermatitis: Role of the skin barrier, environment, microbiome, and therapeutic agents. J Dermatol Sci 2021; 102(3): 142-57). Keratinocytes in AD skin fail to undergo terminal differentiation and are, therefore, prone to the formation of epidermal layers with increased barrier permeability and disturbed composition of lipids and proteins. Importantly, the skin barrier is ensured not simply by just a mixture of lipids and proteins but by an effective complexing and special organization of these molecules. Since no cures for AD are available, primary prevention of AD is an important approach to reduce the prevalence of AD and potentially the atopic march in children at high risk of allergic disease. Nevertheless, most previous interventional trials using emollients from birth to restore skin barrier function failed to prevent the development of AD (Skjerven H O, et al. Skin emollient and early complementary feeding to prevent infant atopic dermatitis (PreventADALL): a factorial, multicentre, cluster-randomised trial. Lancet 2020; 395(10228): 951-61; Bradshaw L E, et al. Emollients for prevention of atopic dermatitis: 5-year findings from the BEEP randomized trial. Allergy 2022; Zhong Y, et al. Emollients in infancy to prevent atopic dermatitis: A systematic review and meta-analysis. Allergy 2022; 77(6): 1685-99). Perhaps it is because of the selection of high-risk for atopy groups into clinical trials; or ingredients of the emollients and the duration of treatment were not appropriate. Or there may need to be additional preventive measures besides the early application of emollients. For effective primary prevention of AD, it is essential to establish a strong set of biomarkers that can identify infants at risk to develop AD. If infants at risk of developing AD before their manifestation of clinical symptoms can be identified, then the application of targeted preemptive intervention with emollients and/or measures to control immune dysregulation may prevent AD development more effectively.

### SUMMARY

One embodiment relates to a method to identify and/or screen a human infant at risk of developing atopic dermatitis (AD), wherein the infant has not been diagnosed as having AD and/or an atopic disease, the method comprising: obtaining a skin sample from the infant, wherein the skin sample is a non-lesional skin sample from the infant; determining from the skin sample an expression level of a cytokine and/or lipid selected from the group consisting of interleukin 13 (IL13), thymic stromal lymphopoietin (TSLP), one or more protein-bound ceramides, one or more sphingomyelins, and a combination thereof; and comparing the level of IL13, TSLP, one or more ceramides, one or more sphingomyelins, or a combination thereof, in the skin sample to a control sample wherein the control sample is from one or more non-atopic (NA) subjects; wherein an elevated level of IL13 and/or TSLP as compared to the control sample identifies the infant as being at risk of developing AD, and/or wherein a decrease in one or more ceramides as compared to the control sample identifies the infant as being at risk of developing AD; and/or wherein an increase in one or more sphingomyelins as compared to the control sample identifies the infant as being at risk of developing AD.

Another embodiment is a method a preemptive intervention in a human infant at risk of developing AD, wherein the infant has not been diagnosed as having AD and/or an atopic disease, the method comprising: obtaining a skin sample from the infant, wherein the skin sample is a non-lesional skin sample from the infant; determining from the skin sample an expression level of a cytokine and/or lipid selected from the group consisting of interleukin 13 (IL13), thymic stromal lymphopoietin (TSLP), one or more ceramides, one or more sphingomyelins, and a combination thereof; and comparing the level of IL13, TSLP, one or more ceramides, one or more sphingomyelins, or a combination thereof, in the skin sample to a control sample wherein the control sample is from one or more non-atopic (NA) subjects; wherein an elevated level of IL13 and/or TSLP as compared to the control sample identifies the infant as being at risk of developing AD, and/or wherein a decrease in one or more ceramides as compared to the control sample identifies the infant as being at risk of developing AD; and/or wherein an increase in one or more sphingomyelins as compared to the control sample identifies the infant as being at risk of developing AD; and providing intervention to the infant identified as being at risk of developing AD, wherein the intervention is selected from the group consisting of administering an immune modifier, a skin barrier enforcing emollient and combinations thereof to the infant.

Another embodiment is a method to prevent and/or decrease the severity of an atopic disease in a human infant at risk of developing AD, wherein the infant has not been diagnosed as having AD and/or an atopic disease, the method comprising: obtaining a skin sample from the infant, wherein the skin sample is a non-lesional skin sample from the infant; determining from the skin sample an expression level of a cytokine and/or lipid selected from the group consisting of interleukin 13 (IL13), thymic stromal lymphopoietin (TSLP), one or more ceramides, one or more sphingomyelins, and a combination thereof; and comparing the level of IL13, TSLP, one or more ceramides, one or more sphingomyelins, or a combination thereof, in the skin sample to a control sample wherein the control sample is from one or more non-atopic (NA) subjects; wherein an elevated level of IL13 and/or TSLP as compared to the control sample identifies the infant as being at risk of developing AD, and/or wherein a decrease in one or more ceramides as compared to the control sample identifies the infant as being at risk of developing AD; and/or wherein an increase in one or more sphingomyelins as compared to the control sample identifies the infant as being at risk of developing AD; and administering to the infant identified as being at risk of developing AD, an immune modifier, a skin barrier enforcing emollient and combinations thereof to the infant.

In one aspect of any of the embodiments related to a method described herein, the ceramides are protein bound ceramides. In one aspect, the protein bound ceramides are ceramides comprising alpha hydroxy fatty acids.

In one aspect of any of the embodiments related to a method described herein, the sphingomyelins are sphingomyelins comprising unsaturated fatty acids.

In one aspect of any of the embodiments related to a method described herein, the immune modifier is selected from the group consisting of an anti-TSLP biologic, an anti-IL4/IL13 biologic and combinations thereof.

In one aspect of any of the embodiments related to a method described herein, the skin sample is obtained by a skin tape stripping method.

In one aspect, the skin tape stripping method comprises: applying an adhesive tape to a target area of the skin of the infant in a manner sufficient to isolate an epidermal sample adhering to the adhesive tape, wherein the epidermal sample comprises cells from the stratum corneum of the infant, wherein the tape comprises a rubber adhesive, and extracting the epidermal sample comprising the one or more proteins adhering to the adhesive tape with a cell scraper comprising thermoplastic elastomer material in a solvent of about 5% to about 30% alcohol in water; and determining in the extracted epidermal sample an expression level of IL13, TSLP, one or more ceramides, one or more sphingomyelins, or a combination thereof.

### BRIEF DESCRIPTION OF THE DRAWINGS

FIG. 1 shows a consort diagram of the study population during the study period as described in the Example section. The number of infants with AD (in brackets) indicates the cumulative number of patients who have developed AD at the time of evaluation.

FIGS. 2A-2C. TSLP and IL13 levels are increased in the Stratum corneum of infants who develop AD later in life. STS samples were collected from the forearm area at the age of 2 months and cytokine determination was performed by MSD assay. Data are shown only for subjects who were fully monitored until the age of 2 years.

FIGS. 3A-3F. Stratum corneum lipid biomarkers as predictors of the future onset of AD. FIG. 3A shows downregulation of the total level and the sums of OS-ceramides with C18-, C20-, and C22-sphingosine. FIG. 3B shows the decrease in the level of individual OS-ceramides with C20-sphingosine molecular species. FIG. 3C shows the lack of decline in the levels of EOS-ceramides, the obligatory precursors of OS-ceramides. FIG. 3D shows the increase in the total and individual levels of sphingomyelins. FIG. 3E shows the increase in the individual levels of AS ceramides with C18-sphingosine. FIG. 3F shows the increase in the ratio between “short-chain” (C14-C22) and “long-chain” (C24-C32) fatty acid-containing NS-ceramides with C18-sphingosine but not with C20- and C22-sphingosine. The p-values represent an unpaired 1-test comparison between healthy and AD groups, n=84 (healthy children), n=27 (future AD children).

FIGS. 4A and 4B. TSLP inhibits the expression of EOS-CER processing enzymes ALOXE2 and ALOX12B in vitro. HEK cells were Ca²⁺-differentiated for seven days then treated with TSLP (10 ng/ml) for 24 hours. The expression of ALOXE3 (FIG. 4A) and ALOX12B (FIG. 4B) was assessed by RT-PCR. Data represents three different experiments each done in duplicate.

FIGS. 5A and 5B. Univariable logistic regression analysis reveals strong combinatorial power of STS cytokines and lipids to predict future onset of AD by 24 months of age.

FIG. 6. The content of filaggrin breakdown products UCA and PCA is the same in the Stratum corneum of future AD and healthy children when assessed at the age of two months.

### DETAILED DESCRIPTION

Atopic dermatitis (AD) is the most common inflammatory skin disease worldwide affecting nearly 30% of children. Very often, AD progresses into food allergy, asthma, and allergic rhinitis that can persist over the entire human life. Collectively, this transition of atopic diseases is called the atopic march. Atopic diseases are impossible or very hard to cure and impose a huge burden on society. Therefore, finding the early biomarkers of the future onset of atopic diseases in newborns, and AD in particular as the first disease in line, opens a window for preemptive interventions that can potentially decrease the severity or totally halt the onset of atopic diseases. Non-invasive collection of biological material to detect biomarkers is critical taking into account the age of infants (early weeks or months), and human SC represent the ideal material that is absolutely benign to collect using skin tape stripping (STS) procedure even on newborns. Until now, there was very limited efforts to search for such biomarkers. Collectively, it was found that at the age of 2 months, SC thymic stromal lymphopoietin (TSLP) predicts the future onset of AD in children from families with a history of atopic diseases, and phytosphingosine is decreased in future AD infants.

The inventors previously identified thymic stromal lymphopoietin (TSLP) as a predictor for the future onset of AD when analyzed in skin tape strips (STS) collected from the forearm of asymptomatic infants at the age of 2 months before the onset of clinical AD (Kim J, et al. Epidermal thymic stromal lymphopoietin predicts the development of atopic dermatitis during infancy. J Allergy Clin Immunol 2016; 137(4): 1282-5 e4). In this earlier work, the inventors pointed out the importance of finding additional predictive biomarkers to strengthen the power of early life STS analyses. To this end, the inventors have undertaken a second study as disclosed herein where newborn infants of Asian ethnicity (infants and their parents were enrolled again in Seoul, Korea) were clinically followed for up to 2 years of age. A non-invasive approach using STS was used to collect Stratum corneum (SC) skin samples from the forearm at the age of 2 months when none of the infants demonstrated any signs of AD and atopic diseases. STS samples were analyzed by liquid chromatography electrospray ionization tandem mass spectrometry (LC-MS/MS) for a wide panel of lipid molecules, and the data were grouped based on clinical diagnosis obtained up to the children's age of 2 years. STS cytokine analysis was performed to gain insight into mechanisms involved in skin barrier function and local immune responses. Such a complex look into skin barrier components allowed the identification of strong novel predicting lipid parameters indicative of the future development of AD and skin barrier failure. This includes protein-bound ceramides that are critical for the scaffold formation during SC maturation and epidermal barrier formation. The inventors also confirmed in this replication cohort that TSLP is upregulated at 2 months of age in future AD subjects. In addition, the inventors determined simultaneous upregulation of interleukin (IL) 13, a byproduct of TSLP activation, and also demonstrated the ability of TSLP to inhibit in vitro the expression of enzymes that control protein-bound ceramide formation. Finally, a combination of type 2 cytokines and lipid biomarkers in the skin increased the odds ratio (OR) of future AD development prediction to >50. These results not only provide a method to screen infants at high risk of developing AD but also provide a rationale for establishing preventive measures such as applying emollients containing ceramides or introducing biologics or other therapeutic options that diminish T helper 2 skewed immune status.

The findings presented herein confirm that at the age of 2 months, before the onset of clinical AD, TSLP is a good predictor of the future onset of AD, demonstrates that interleukin 13 is even better than TSLP at predicting future AD, and identifies novel lipid biomarkers that predict future onset of AD with high power. Within novel lipid biomarkers, loss of protein-bound ceramides is of special importance as protein-bound ceramides are critical for the ultrastructural organization of the lamellae and skin barrier function in general. Within other lipid biomarkers, a decline in ceramides with alpha-hydroxy fatty acids and an increase in sphingomyelins with unsaturated fatty acids are of the most significance. Together, a combination of cytokine and lipid biomarkers were found to predict the future onset of AD with the odds ratio of 51.3. Indeed, the odds ratio of 51.3 is surprisingly high, as can be seen from the results of the previous study in which the odds ratio of filaggrin mutation for AD development was only 3.1. The finding presented herein also found that TSLP blocks the expression of ALOXE3 and ALOX12B enzymes that oxidize protein-bound ceramide precursors, thus providing a mechanistic explanation for their decline in SC of future AD infants.

The findings herein provide a roadmap for preemptive intervention in infants at risk of future development of AD: (1) screening high-risk infants using SC biomarkers, and (2) intervention by targeting the involved cytokines with immune modifiers, such as biologics, and skin barrier enforcing emollients. This strategy is believed to be able to prevent the development of early-life AD or significantly reduce the severity of eczema and dramatically change current pediatric practice.

The inventors have previously demonstrated (Kim J, et al. Epidermal thymic stromal lymphopoietin predicts the development of atopic dermatitis during infancy. J Allergy Clin Immunol 2016; 137(4): 1282-5 e4) and experimental models in other laboratories (Ziegler S F. Thymic stromal lymphopoietin, skin barrier dysfunction, and the atopic march. Ann Allergy Asthma Immunol 2021; 127(3): 306-11), the role of skin TSLP in AD development and association of high TSLP levels in the SC of infants with future risk of AD. Considering that AD usually precedes other atopic diseases such as food allergy, asthma, and allergic rhinitis, early prediction of future AD development is of utmost importance as it indicates the time for active preemptive intervention. The current work on a separate cohort of children not only confirmed the inventor's earlier observation but also found for the first time that protein-bound OS ceramides as well as unsaturated sphingomyelins and several free ceramide molecules in extracellular matrix were altered as early as 2 months of age in the skin of infants who developed AD later. More importantly, these results highlight novel skin biomarkers of the future AD onset that, when combined, provide an unprecedented power of prediction and open the field of precision medicine in AD.

The current findings presented herein demonstrate that at the age of 2 months, skin keratinocytes are already challenged to produce TSLP that, in turn, stimulates lymphocytes for type 2 cytokine production. Importantly, TSLP was found to significantly downregulate ALOXE3 and ALOX12B in human keratinocytes that are critical for EOS-CER to OS-CER transformation and subsequent binding to proteins in the cornified envelope. The lack of OS-CER binding to involucrin and periplakin, major binding partners of OS-CER in the cornified envelope, (Nemes Z, et al. A novel function for transglutaminase 1: attachment of long-chain omega-hydroxyceramides to involucrin by ester bond formation. Proc Natl Acad Sci USA 1999; 96(15): 8402-7; and Marekov L N, Steinert P M. Ceramides are bound to structural proteins of the human foreskin epidermal cornified cell envelope. J Biol Chem 1998; 273(28): 17763-70) is clinically important because it results in disorganized lamellae formation and skin barrier dysfunction. The upregulation of TSLP and IL13 levels in the SC also explains the overall shortening of fatty acids in NS- and AS-CERs, because IL13 and IL4 block fatty acid elongation through the inhibition of ELOVL3 and ELOV6 expression (Berdyshev E, et al. Lipid abnormalities in atopic skin are driven by type 2 cytokines. JCI Insight 2018; 3(4)).

Of note, compared to a recent study by Rinnov et al., (Rinnov M R, et al. Skin biomarkers predict development of atopic dermatitis in infancy. Allergy 2022) the present findings are more specific to AD in that TSLP and IL13 levels in the SC were found higher in infants who developed AD later. The present findings also did not find the downregulation of phytosphingosine level in SC of future AD infants (Table 1 below) reported by Rinnov et al. Furthermore, in future AD infants, the findings provided herein found the increase in monounsaturated sphingomyelins that disrupt the lamellae structure (Nishifuji K, Yoon J S. The Stratum corneum: the rampart of the mammalian body. Vet Dermatol 2013; 24(1): 60-72 e15-6). Altogether, the findings provided herein indicate that alterations of SC lipids as early as at 2 months of age contribute to the onset of clinical AD later in their life.

TABLE 1
Amounts of lipids in the stratum corneum of healthy and
future AD children collected at the age of two months¹

| | | Amount (pmol/mg protein) | | | | |
|---|---|---|---|---|---|---|
| Lipid group and | | NA | | AD | | |
| Common name | Abbreviation | Average | SEM | Average | SEM | p-value |
| Sphingomyelins | | | | | | |
| SM(d18:1/16:0) | 16:0-SM | 20.4 | 2.16 | 31.1 | 6.36 | 0.054 |
| SM(d18:1/17:0) | 17:0-SM | 1.4 | 0.09 | 2.4 | 0.50 | 0.003 |
| SM(d18:1/18:0) | 18:0-SM | 11.0 | 1.40 | 13.3 | 2.33 | 0.430 |
| SM(d18:1/20:0) | 20:0-SM | 5.3 | 0.63 | 6.0 | 0.80 | 0.617 |
| SM(d18:1/22:0) | 22:0-SM | 11.8 | 1.16 | 13.6 | 1.39 | 0.442 |
| SM(d18:1/24:1) | 24:1-SM | 5.1 | 0.84 | 21.4 | 4.52 | 5.04E−07 |
| SM(d18:1/16:0) | 16:0-SM | 20.4 | 2.16 | 31.1 | 6.36 | 0.054 |
| SM(d18:1/17:0) | 17:0-SM | 1.4 | 0.09 | 2.4 | 0.50 | 0.003 |
| SM(d18:1/18:0) | 18:0-SM | 11.0 | 1.40 | 13.3 | 2.33 | 0.430 |
| SM(d18:1/20:0) | 20:0-SM | 5.3 | 0.63 | 6.0 | 0.80 | 0.617 |
| SM(d18:1/22:0) | 22:0-SM | 11.8 | 1.16 | 13.6 | 1.39 | 0.442 |
| SM(d18:1/24:1) | 24:1-SM | 5.1 | 0.84 | 21.4 | 4.52 | 5.04E−07 |
| SM(d18:1/16:0) | 16:0-SM | 20.4 | 2.16 | 31.1 | 6.36 | 0.054 |
| SM(d18:1/17:0) | 17:0-SM | 1.4 | 0.09 | 2.4 | 0.50 | 0.003 |
| SM(d18:1/18:0) | 18:0-SM | 11.0 | 1.40 | 13.3 | 2.33 | 0.430 |
| SM(d18:1/20:0) | 20:0-SM | 5.3 | 0.63 | 6.0 | 0.80 | 0.617 |
| Sphingoid Bases | | | | | | |
| Sphingosine | Sph | 241.7 | 11.43 | 302.4 | 22.83 | 0.016 |
| Sphinganine | DHSph | 86.4 | 6.53 | 95.7 | 5.84 | 0.462 |
| C17-Sphingosine | C17-Sph | 46.2 | 1.97 | 58.8 | 3.92 | 0.004 |
| C17-Sphinganine | C17-DHSph | 11.4 | 1.80 | 11.2 | 2.94 | 0.960 |
| Phytosphingosine | PhytoSPH | 249.2 | 46.73 | 231.4 | 36.28 | 0.842 |
| C19-Sphingosine | C19-Sph | 60.3 | 3.14 | 75.1 | 5.73 | 0.029 |
| C19-Sphinganine | C19-DHSph | 18.8 | 2.42 | 25.0 | 2.13 | 0.183 |
| C20-Sphingosine | C20-Sph | 563.1 | 32.66 | 624.0 | 55.80 | 0.367 |
| C20-Sphinganine | C20-DHSph | 131.6 | 7.12 | 151.5 | 12.32 | 0.181 |
| C21-Sphingosine | C21-Sph | 92.1 | 6.21 | 105.8 | 10.13 | 0.282 |
| C21-Sphinganine | C21-DHSph | 47.4 | 2.26 | 54.0 | 3.73 | 0.156 |
| C22-Sphingosine | C22-Sph | 283.7 | 15.89 | 311.3 | 28.11 | 0.404 |
| C22-Sphinganine | C22-DHSph | 623.7 | 27.78 | 714.2 | 59.62 | 0.134 |
| EOS-Ceramides | | | | | | |
| omega-linoleoyloxy- | EO26S18-CER | 1.2 | 0.05 | 1.5 | 0.16 | 0.042 |
| Cer(d18:1/26:0) | | | | | | |
| omega-linoleoyloxy- | EO28S18-CER | 18.7 | 0.74 | 24.0 | 2.20 | 0.005 |
| Cer(d18:1/28:0) | | | | | | |
| omega-linoleoyloxy- | EO30S18-CER | 238.1 | 8.95 | 288.0 | 23.75 | 0.018 |
| Cer(d18:1/30:0) | | | | | | |
| omega-linoleoyloxy- | EO32S18-CER | 86.7 | 3.46 | 99.3 | 7.89 | 0.097 |
| Cer(d18:1/32:0) | | | | | | |
| omega-linoleoyloxy- | EO34S18-CER | 6.9 | 0.30 | 7.6 | 0.73 | 0.364 |
| Cer(d18:1/34:0) | | | | | | |
| omega-linoleoyloxy- | EO26S20-CER | 3.9 | 0.17 | 4.6 | 0.36 | 0.038 |
| Cer(d20:1/26:0) | | | | | | |
| omega-linoleoyloxy- | EO28S20-CER | 71.8 | 3.09 | 79.7 | 6.03 | 0.240 |
| Cer(d20:1/28:0) | | | | | | |
| omega-linoleoyloxy- | EO30S20-CER | 786.4 | 30.63 | 813.5 | 51.89 | 0.663 |
| Cer(d20:1/30:0) | | | | | | |
| omega-linoleoyloxy- | EO32S20-CER | 252.9 | 10.26 | 249.9 | 16.48 | 0.885 |
| Cer(d20:1/32:0) | | | | | | |
| omega-linoleoyloxy- | EO34S20-CER | 14.9 | 0.67 | 13.1 | 1.05 | 0.177 |
| Cer(d20:1/34:0) | | | | | | |
| omega-linoleoyloxy- | EO26S22-CER | 3.4 | 0.14 | 3.6 | 0.24 | 0.570 |
| Cer(d22:1/26:0) | | | | | | |
| omega-linoleoyloxy- | EO28S22-CER | 44.5 | 1.80 | 42.2 | 2.96 | 0.533 |
| Cer(d22:1/28:0) | | | | | | |
| omega-linoleoyloxy- | EO30S22-CER | 400.3 | 14.27 | 357.2 | 19.49 | 0.135 |
| Cer(d22:1/30:0) | | | | | | |
| omega-linoleoyloxy- | EO32S22-CER | 107.3 | 4.43 | 91.1 | 5.45 | 0.066 |
| Cer(d22:1/32:0) | | | | | | |
| omega-linoleoyloxy- | EO34S22-CER | 5.5 | 0.28 | 4.1 | 0.31 | 0.009 |
| Cer(d22:1/34:0) | | | | | | |
| N(C18S)-Ceramides | | | | | | |
| Cer(d18:1/14:0) | 14:0-C18S-CER | 4.8 | 0.42 | 4.6 | 0.56 | 0.837 |
| Cer(d18:1/16:0) | 16:0-C18S-CER | 79.1 | 4.57 | 148.7 | 29.78 | 0.001 |
| Cer(d18:1/17:0) | 17:0-C18S-CER | 7.1 | 0.35 | 11.5 | 2.18 | 0.003 |
| Cer(d18:1/18:0) | 18:0-C18S-CER | 19.5 | 0.99 | 24.4 | 3.33 | 0.069 |
| Cer(d18:1/20:0) | 20:0-C18S-CER | 8.8 | 0.40 | 12.3 | 2.09 | 0.016 |
| Cer(d18:1/22:1) | 22:1-C18S-CER | 8.0 | 0.38 | 10.1 | 1.13 | 0.029 |
| Cer(d18:1/22:0) | 22:0-C18S-CER | 37.6 | 1.67 | 42.3 | 4.73 | 0.260 |
| Cer(d18:1/24:1(15Z)) | 24:1-C18S-CER | 88.9 | 4.23 | 96.2 | 7.40 | 0.414 |
| Cer(d18:1/24:0) | 24:0-C18S-CER | 205.6 | 12.72 | 242.8 | 15.54 | 0.144 |
| Cer(d18:1/26:1(17Z)) | 26:1-C18S-CER | 138.1 | 6.77 | 133.0 | 8.59 | 0.706 |
| Cer(d18:1/26:0) | 26:0-C18S-CER | 329.5 | 15.93 | 306.2 | 21.39 | 0.466 |
| Cer(d18:1/28:0) | 28:0-C18S-CER | 123.1 | 5.76 | 121.0 | 7.70 | 0.858 |
| Cer(d18:1/30:0) | 30:0-C18S-CER | 23.8 | 1.37 | 26.3 | 2.06 | 0.378 |
| Cer(d18:1/32:0) | 32:0-C18S-CER | 1.7 | 0.13 | 2.0 | 0.21 | 0.223 |
| | Total N(C18S)-CER | 1075.4 | 47.77 | 1181.4 | 85.66 | 0.301 |
| N(C20S)-Ceramides | | | | | | |
| Cer(d20:1/14:0) | 14:0-C20S-CER | 5.3 | 0.44 | 4.1 | 0.61 | 0.178 |
| Cer(d20:1/16:0) | 16:0-C20S-CER | 18.4 | 1.12 | 18.0 | 1.86 | 0.884 |
| Cer(d20:1/18:0) | 18:0-C20S-CER | 20.4 | 0.91 | 19.6 | 1.36 | 0.670 |
| Cer(d20:1/20:0) | 20:0-C20S-CER | 14.1 | 0.62 | 12.8 | 0.90 | 0.313 |
| Cer(d20:1/22:0) | 22:0-C20S-CER | 46.9 | 2.17 | 39.2 | 2.64 | 0.074 |
| Cer(d20:1/24:1(15Z)) | 24:1-C20S-CER | 102.7 | 5.66 | 74.5 | 3.55 | 0.009 |
| Cer(d20:1/24:0) | 24:0-C20S-CER | 259.5 | 12.68 | 220.1 | 12.86 | 0.108 |
| Cer(d20:1/26:1(17Z)) | 26:1-C20S-CER | 146.5 | 7.53 | 126.8 | 6.58 | 0.176 |
| Cer(d20:1/26:0) | 26:0-C20S-CER | 402.1 | 19.11 | 364.1 | 23.06 | 0.314 |
| Cer(d20:1/28:0) | 28:0-C20S-CER | 387.5 | 16.99 | 410.4 | 25.34 | 0.513 |
| Cer(d20:1/30:0) | 30:0-C20S-CER | 89.2 | 4.99 | 97.0 | 7.96 | 0.455 |
| Cer(d20:1/32:0) | 32:0-C20S-CER | 5.1 | 0.42 | 5.6 | 0.58 | 0.547 |
| | Total N(C20S)-CER | 1497.7 | 66.77 | 1392.3 | 72.27 | 0.421 |
| N(C22S)-Ceramides | | | | | | |
| Cer(d22:1/14:0) | 14:0-C22S-CER | 0.5 | 0.06 | 0.3 | 0.06 | 0.129 |
| Cer(d22:1/16:0) | 16:0-C22S-CER | 3.8 | 0.24 | 3.6 | 0.32 | 0.745 |
| Cer(d22:1/18:0) | 18:0-C22S-CER | 24.3 | 0.96 | 19.9 | 0.90 | 0.018 |
| Cer(d22:1/20:0) | 20:0-C22S-CER | 7.8 | 0.33 | 5.8 | 0.35 | 0.002 |
| Cer(d22:1/22:0) | 22:0-C22S-CER | 22.0 | 1.00 | 16.5 | 0.98 | 0.005 |
| Cer(d22:1/24:1(15Z)) | 24:1-C22S-CER | 36.9 | 1.89 | 27.5 | 1.64 | 0.011 |
| Cer(d22:1/24:0) | 24:0-C22S-CER | 117.5 | 5.37 | 101.5 | 6.40 | 0.134 |
| Cer(d22:1/26:1(17Z)) | 26:1-C22S-CER | 63.2 | 3.52 | 49.1 | 3.13 | 0.040 |
| Cer(d22:1/26:0) | 26:0-C22S-CER | 248.7 | 11.36 | 234.3 | 16.46 | 0.536 |
| Cer(d22:1/28:0) | 28:0-C22S-CER | 311.0 | 15.01 | 299.6 | 20.23 | 0.707 |
| Cer(d22:1/30:0) | 30:0-C22S-CER | 122.5 | 6.71 | 113.1 | 8.61 | 0.483 |
| Cer(d22:1/32:0) | 32:0-C22S-CER | 4.6 | 0.36 | 4.4 | 0.47 | 0.769 |
| | Total N(C22S)-CER | 962.8 | 44.30 | 875.7 | 53.06 | 0.323 |
| N(C18DS)-Ceramides | | | | | | |
| Cer(d18:0/16:0) | 16:0-C18DS-CER | 26.9 | 1.66 | 34.6 | 4.07 | 0.051 |
| Cer(d18:0/18:0) | 18:0-C18DS-CER | 7.5 | 0.50 | 6.8 | 0.86 | 0.524 |
| Cer(d18:0/20:0) | 20:0-C18DS-CER | 14.5 | 0.69 | 17.0 | 1.67 | 0.126 |
| Cer(d18:0/22:0) | 22:0-C18DS-CER | 37.5 | 1.21 | 37.6 | 2.19 | 0.972 |
| Cer(d18:0/24:0) | 24:0-C18DS-CER | 131.2 | 4.96 | 127.8 | 8.32 | 0.743 |
| Cer(d18:0/26:0) | 26:0-C18DS-CER | 94.8 | 4.05 | 87.2 | 8.34 | 0.378 |
| Cer(d18:0/28:0) | 28:0-C18DS-CER | 31.6 | 1.43 | 34.2 | 3.42 | 0.421 |
| | Total N(C18DS)-CER | 343.9 | 12.23 | 345.2 | 22.54 | 0.961 |
| N(C20DS)-Ceramides | | | | | | |
| Cer(d20:0/16:0) | 16:0-C20DS-CER | 1.7 | 0.17 | 2.1 | 0.23 | 0.162 |
| Cer(d20:0/18:0) | 18:0-C20DS-CER | 14.3 | 0.75 | 17.7 | 1.73 | 0.045 |
| Cer(d20:0/20:0) | 20:0-C20DS-CER | 19.0 | 1.14 | 15.0 | 1.36 | 0.075 |
| Cer(d20:0/22:0) | 22:0-C18DS-CER | 22.9 | 1.09 | 19.8 | 1.52 | 0.164 |
| Cer(d20:0/24:0) | 24:0-C20DS-CER | 126.6 | 6.46 | 115.6 | 9.24 | 0.404 |
| Cer(d20:0/26:0) | 26:0-C20DS-CER | 115.0 | 6.02 | 118.8 | 11.58 | 0.774 |
| Cer(d20:0/28:0) | 28:0-C20DS-CER | 32.6 | 1.86 | 37.4 | 3.99 | 0.259 |
| | Total N(C20DS)-CER | 332.2 | 16.06 | 326.5 | 26.94 | 0.866 |
| N(C22DS)-Ceramides | | | | | | |
| Cer(d22:0/16:0) | 16:0-C22DS-CER | 3.8 | 0.25 | 4.3 | 0.34 | 0.319 |
| Cer(d22:0/18:0) | 18:0-C22DS-CER | 57.9 | 2.36 | 52.9 | 2.72 | 0.282 |
| Cer(d22:0/20:0) | 20:0-C22DS-CER | 26.1 | 1.32 | 20.5 | 1.42 | 0.032 |
| Cer(d22:0/22:0) | 22:0-C22DS-CER | 32.7 | 1.34 | 29.5 | 2.19 | 0.257 |
| Cer(d22:0/24:0) | 24:0-C22DS-CER | 153.4 | 6.26 | 145.8 | 12.34 | 0.577 |
| Cer(d22:0/26:0) | 26:0-C22DS-CER | 109.0 | 4.98 | 106.5 | 9.78 | 0.822 |
| Cer(d22:0/28:0) | 28:0-C22DS-CER | 26.6 | 1.38 | 25.5 | 2.68 | 0.706 |
| | Total N(C22DS)-CER | 409.4 | 16.14 | 384.8 | 29.32 | 0.479 |
| Ratio ΣEO (C18S)- | | 0.4 | 0.02 | 0.383 | 0.03 | 0.473 |
| CER/ΣN(C18S)-CER | | | | | | |
| Ratio ΣEO(C20S)- | | 0.8 | 0.03 | 0.880 | 0.06 | 0.288 |
| CER/ΣN(C20S)-CER | | | | | | |
| Ratio ΣEO(C22S)- | | 0.6 | 0.02 | 0.613 | 0.04 | 0.721 |
| CER/ΣN(C22S)-CER | | | | | | |
| Protein-Bound | | | | | | |
| OS-Ceramides | | | | | | |
| Cer(d18:1/28:0(26OH)) | 26:0-O-C18S-CER | 9.6 | 1.38 | 5.4 | 1.29 | 0.120 |
| Cer(d18:1/28:0(28OH)) | 28:0-O-C18S-CER | 156.2 | 6.13 | 127.4 | 10.25 | 0.024 |
| Cer(d18:1/30:0(30OH)) | 30:0-O-C18S-CER | 1524.5 | 57.20 | 1160.7 | 116.17 | 0.003 |
| Cer(d18:1/32:0(32OH)) | 32:0-O-C18S-CER | 344.4 | 13.81 | 255.9 | 29.21 | 0.003 |
| Cer(d20:1/28:0(26OH)) | 26:0-O-C20S-CER | 16.8 | 0.70 | 16.3 | 1.21 | 0.739 |
| Cer(d20:1/28:0(28OH)) | 28:0-O-C20S-CER | 315.8 | 15.22 | 246.5 | 26.56 | 0.031 |
| Cer(d20:1/30:0(30OH)) | 30:0-O-C20S-CER | 2105.4 | 89.11 | 1421.4 | 180.27 | 3.92E−04 |
| Cer(d20:1/32:0(32OH)) | 32:0-O-C20S-CER | 402.2 | 17.04 | 267.7 | 35.98 | 2.22E−04 |
| Cer(d22:1/28:0(26OH)) | 26:0-O-C22S-CER | 2.3 | 0.14 | 2.4 | 0.25 | 0.823 |
| Cer(d22:1/28:0(28OH)) | 28:0-O-C22S-CER | 35.4 | 1.75 | 25.1 | 3.03 | 0.005 |
| Cer(d22:1/30:0(30OH)) | 30:0-O-C22S-CER | 320.1 | 14.31 | 193.8 | 25.28 | 3.29E−05 |
| Cer(d22:1/32:0(32OH)) | 32:0-O-C22S-CER | 58.5 | 2.66 | 36.7 | 5.05 | 1.00E−04 |
| | Total OS-CER | 5291.4 | 203.44 | 3759 | 414.24 | 0.001 |
| | Total O(C18S)-CER | 2034.8 | 74.46 | 1549 | 150.39 | 0.003 |
| | Total O(C20S)-CER | 2840.2 | 117.38 | 1952 | 236.83 | 4.53E−04 |
| A(C18S)-Ceramides | | | | | | |
| Cer(d18:1(4E)/16:0(2OH)) | A16:0-C18S-CER | 390.3 | 15.32 | 538.4 | 65.04 | 0.002 |
| Cer(d18:1(4E)/18:0(2OH)) | A18:0-C18S-CER | 165.3 | 8.71 | 212.6 | 19.24 | 0.017 |
| Cer(d18:1(4E)/20:0(2OH)) | A20:0-C18S-CER | 13.5 | 0.69 | 17.9 | 2.44 | 0.021 |
| Cer(d18:1(4E)/22:0(2OH)) | A22:0-C18S-CER | 30.5 | 1.50 | 47.7 | 5.14 | 4.95E−05 |
| Cer(d18:1(4E)/24:0(2OH)) | A24:0-C18S-CER | 594.9 | 27.80 | 673.5 | 63.90 | 0.219 |
| Cer(d18:1(4E)/26:0(2OH)) | A26:0-C18S-CER | 759.1 | 37.84 | 821.9 | 64.52 | 0.433 |
| Cer(d18:1(4E)/28:0(2OH)) | A28:0-C18S-CER | 37.1 | 1.91 | 40.5 | 2.66 | 0.391 |
| | Total A(C18S)-CER | 1990.7 | 83.14 | 2353 | 204.07 | 0.066 |
| A(C20S)-Ceramides | | | | | | |
| Cer(d20:1(4E)/16:0(2OH)) | A16:0-C20S-CER | 251.7 | 14.90 | 305.9 | 24.25 | 0.081 |
| Cer(d20:1(4E)/18:0(2OH)) | A18:0-C20S-CER | 39.0 | 1.99 | 43.4 | 3.15 | 0.279 |
| Cer(d20:1(4E)/20:0(2OH)) | A20:0-C20S-CER | 12.6 | 0.64 | 17.9 | 1.32 | 2.21E−04 |
| Cer(d20:1(4E)/22:0(2OH)) | A22:0-C20S-CER | 32.0 | 1.96 | 28.5 | 2.01 | 0.365 |
| Cer(d20:1(4E)/24:0(2OH)) | A24:0-C20S-CER | 813.1 | 44.56 | 772.4 | 47.54 | 0.640 |
| Cer(d20:1(4E)/26:0(2OH)) | A26:0-C20S-CER | 1134.6 | 65.60 | 1068.9 | 54.47 | 0.602 |
| Cer(d20:1(4E)/28:0(2OH)) | A28:0-C20S-CER | 104.9 | 5.99 | 116.2 | 7.35 | 0.341 |
| | Total A(C20S)-CER | 2387.9 | 126.06 | 2353 | 120.24 | 0.887 |
| A(C22S)-Ceramides | | | | | | |
| Cer(d22:1(4E)/16:0(2OH)) | A16:0-C22S-CER | 23.4 | 1.17 | 24.8 | 1.45 | 0.546 |
| Cer(d22:1(4E)/18:0(2OH)) | A18:0-C22S-CER | 28.9 | 1.47 | 35.3 | 1.88 | 0.030 |
| Cer(d22:1(4E)/20:0(2OH)) | A20:0-C22S-CER | 5.8 | 0.30 | 5.2 | 0.41 | 0.302 |
| Cer(d22:1(4E)/22:0(2OH)) | A22:0-C22S-CER | 11.2 | 0.66 | 10.1 | 0.64 | 0.421 |
| Cer(d22:1(4E)/24:0(2OH)) | A24:0-C22S-CER | 240.4 | 13.53 | 207.4 | 11.04 | 0.205 |
| Cer(d22:1(4E)/26:0(2OH)) | A26:0-C22S-CER | 285.0 | 17.53 | 262.2 | 18.64 | 0.506 |
| Cer(d22:1(4E)/28:0(2OH)) | A28:0-C22S-CER | 27.9 | 1.78 | 28.6 | 2.35 | 0.859 |
| | Total A(C22S)-CER | 622.6 | 34.76 | 574 | 31.66 | 0.466 |
| N(C18P)-Ceramides | | | | | | |
| Cer(t18:0/16:0) | 16:0-C18P-CER | 29.7 | 4.35 | 33.9 | 4.93 | 0.627 |
| Cer(t18:0/18:0) | 18:0-C18P-CER | 119.7 | 20.65 | 140.4 | 26.66 | 0.618 |
| Cer(t18:0/20:0) | 20:0-C18P-CER | 22.1 | 1.38 | 22.7 | 2.36 | 0.818 |
| Cer(t18:0/22:0) | 22:0-C18P-CER | 122.5 | 6.38 | 146.2 | 9.03 | 0.068 |
| Cer(t18:0/24:0) | 24:0-C18P-CER | 2205.5 | 107.54 | 1845.9 | 155.01 | 0.099 |
| Cer(t18:0/26:0) | 26:0-C18P-CER | 2276.9 | 114.72 | 1794.9 | 182.00 | 0.035 |
| Cer(t18:0/28:0) | 28:0-C18P-CER | 742.4 | 36.24 | 597.6 | 48.55 | 0.043 |
| Cer(t18:0/30:0) | 30:0-C18P-CER | 112.6 | 6.17 | 97.1 | 7.95 | 0.203 |
| Cer(t18:0/32:0) | 32:0-C18P-CER | 8.1 | 0.84 | 7.3 | 1.08 | 0.648 |
| | Total N(C18P)-CER | 5639.5 | 270.21 | 4686.1 | 386.62 | 0.077 |
| N(C20P)-Ceramides | | | | | | |
| Cer(t20:0/16:0) | 16:0-C20P-CER | 37.3 | 2.54 | 41.7 | 3.58 | 0.389 |
| Cer(t20:0/18:0) | 18:0-C20P-CER | 13.2 | 0.90 | 11.6 | 1.52 | 0.413 |
| Cer(t20:0/20:0) | 20:0-C20P-CER | 14.7 | 0.85 | 20.0 | 2.25 | 0.010 |
| Cer(t20:0/22:0) | 22:0-C20P-CER | 64.6 | 3.15 | 62.8 | 5.43 | 0.779 |
| Cer(t20:0/24:0) | 24:0-C20P-CER | 1064.6 | 50.15 | 889.1 | 68.71 | 0.080 |
| Cer(t20:0/26:0) | 26:0-C20P-CER | 1197.1 | 61.17 | 959.4 | 73.40 | 0.047 |
| Cer(t20:0/28:0) | 28:0-C20P-CER | 403.0 | 20.49 | 368.2 | 21.61 | 0.386 |
| Cer(t20:0/30:0) | 30:0-C20P-CER | 72.0 | 4.18 | 74.8 | 5.37 | 0.739 |
| | Total N(C20P)-CER | 2866.4 | 137.84 | 2427.7 | 164.75 | 0.105 |
| N(C22P)-Ceramides | | | | | | |
| Cer(t22:0/16:0) | 16:0-C22P-CER | 12.7 | 1.54 | 10.0 | 1.02 | 0.352 |
| Cer(t22:0/18:0) | 18:0-C22P-CER | 34.5 | 2.16 | 39.2 | 5.50 | 0.365 |
| Cer(t20:0/20:0) | 20:0-C22P-CER | 36.3 | 1.67 | 32.8 | 2.44 | 0.296 |
| Cer(t22:0/22:0) | 22:0-C22P-CER | 138.5 | 6.24 | 115.9 | 7.16 | 0.066 |
| Cer(t22:0/24:0) | 24:0-C22P-CER | 1768.0 | 79.59 | 1452.3 | 94.55 | 0.045 |
| Cer(t22:0/26:0) | 26:0-C22P-CER | 1616.2 | 75.82 | 1408.8 | 86.20 | 0.165 |
| Cer(t22:0/28:0) | 28:0-C22P-CER | 517.4 | 26.06 | 503.7 | 28.84 | 0.790 |
| Cer(t22:0/30:0) | 30:0-C22P-CER | 81.3 | 4.58 | 75.6 | 5.06 | 0.524 |
| | Total N(C22P)-CER | 4204.9 | 189.58 | 3638.3 | 210.66 | 0.128 |

¹N(C18-C22S)S-CER were quantitated against 16:0-((d7)C18S)-CER and correction coefficients from standard curves created by mixing fixed amount of internal standard and variable amounts of 14:0-24:0-(C18S)-ceramides.
N(C18-C22DS)-CER were quantitated against 16:0-((d7)C18S)-CER and correction coefficients from standard curves created by mixing fixed amount of internal standard and variable amounts of 14:0-24:0-(C18DS)-ceramides.
A(C18-C22S)-CER and O(C18-C22S)-CER were analyzed semiquantitativelyby directly comparing the signal from the internal standard (CER5(d9), N-(2′-(R)-hydroxypalmitoyl(d9)) D-erythro-sphingosine) with the signal of each detected AS-CER and OS-CER molecule.
N(C18-C22P)-CER were analyzed semiquantitativelyby directly comparing the signal from the internal standard (CER6-2′S(d9), N-(2′-(S)-hydroxypalmitoyl(d9)) D-ribo-phytosphingosine) with the signal of each detected NP-CER molecule.
EO(C18-C22S)-CER were analyzed semiquantitativelyby directly comparing the signal from the internal standard (CER1(d9), (d18:1/26:0/18:1(d9))) with the signal of each detected EOS-CER molecule.
Sphingomyelins were quantitated against N12:0-sphingomyelin and correction coefficients from standard curves created by mixing fixed amount of internal standard and variable amounts of N14:0-24:0-sphingomyelins.
Sphingoidbases Sphand DHSphwere quantitated against (d7)C18-Sphingosine and correction coefficients from standard curves created by mixing fixed amount of internal standard and variable amounts of C17-C18(dihydro)sphingosines and C18-phytosphingosine. Same coefficients were used for calculation of amounts of C19-C22 (DH)Sph.

In the results presented herein, no abnormalities in the level of FLG breakdown products UCA and PCA between healthy infants and infants that have developed AD in the future were found. FLG plays a critical role in assembling a scaffold for keratins for the cornified envelop formation (Candi E, et al. The cornified envelope: a model of cell death in the skin. Nat Rev Mol Cell Biol 2005; 6(4): 328-40) and multiple mutations in FLG gene are associated with higher incidence of AD development (Leung D Y M, et al. Cutaneous barrier dysfunction in allergic diseases. J Allergy Clin Immunol 2020; 145(6): 1485-97; Drislane C, Irvine A D. The role of filaggrin in atopic dermatitis and allergic disease. Ann Allergy Asthma Immunol 2020; 124(1): 36-43). IL4 and IL13 also inhibit FLG expression (Howell M D, Kim B E, Gao P, et al. Cytokine modulation of atopic dermatitis filaggrin skin expression. J Allergy Clin Immunol 2007; 120(1): 150-5). However, at the age of 2 months, the influence of type 2 cytokines may not be sufficient to affect FLG transcription and FLG processing or is too early to be detected that, at the end, is demonstrated by normal levels of FLG breakdown products UCA and PCA.

Not undermining the importance of immunological markers of the future onset of AD, the most significant result is the discovery of novel lipid biomarkers of a distant onset of AD that carried a substantial predictive power, especially when assessed together with TSLP or IL13. Changes in several lipid molecules, even assessed alone, allowed to predict future onset of AD with ORs between 3.5 and 17.5. However, a combined power of prediction using changes in the levels of type 2 cytokines, OS-CER, and unsaturated SM or AS-CER reached an unprecedented OR values between 41 and 51. Indeed, the OR of 51.3 is surprisingly high, as can be seen from the results of the previous study in which the OR of FLG mutation for AD development was only 3.1 (Irvine A D, et al. Filaggrin mutations associated with skin and allergic diseases. N Engl J Med 2011; 365(14): 1315-27). This grants the ability to identify children at risk of future AD onset with high precision. Taking into account that targeted anti-TSLP (Kurihara M, et al. Current summary of clinical studies on anti-TSLP antibody, Tezepelumab, in asthma. Allergol Int 2022) and anti-IL4/IL13 (Koskeridis F, et al. Treatment With Dupilumab in Patients With Atopic Dermatitis: Systematic Review and Meta-Analysis. J Cutan Med Surg 2022; 26(6): 613-21; Kelly K A, et al. Therapeutic Potential of Tralokinumab in the Treatment of Atopic Dermatitis: A Review on the Emerging Clinical Data. Clin Cosmet Investig Dermatol 2022; 15: 1037-43) biologics are now available, and one of them (dupilumab) is already approved for children as young as 6 months of age, a window for targeted precision medicine is opening with a possibility to change the entire pediatric practice. More studies are needed to clarify if found lipid and cytokine changes can be used to predict future onset of AD in children from different ethnical groups. Non-invasive method of SC collection using STS is safe to use even on a skin of newborn children that, together with a possibility to use targeted biologics, brings strong expectation that atopic diseases and the atopic march can be prevented.

An “individual” is a vertebrate, such as a mammal, including without limitation a human. Mammals include, but are not limited to, farm animals, sport animals, pets, primates, mice and rats. The term “individual” can be used interchangeably with the term “animal”, “subject” or “patient”.

In one aspect of the invention, the subject is human. In one aspect, the subject is a child (less than 18 years of age). In another aspect, the subject is an infant. Infant as used herein is defined as up to two years (24 months) of age. In addition, an asymptomatic subject, is a subject that is not producing or showing symptoms of an allergic disease. For example, an AD asymptomatic subject is a subject that is not producing or showing symptoms of AD such as, itching, red patches on the skin (especially on the hands, feet, ankles, wrists, neck, upper chest, eyelids, inside the bend of the elbows and knees, face and scalp); small, raised bumps which can leak fluid and crust over when scratched; thickened, cracked, dry, scaly skin; and raw, sensitive, swollen skin from scratching. Most often, AD begins before age 5 and may persist into adolescence and adulthood. For some AD subjects, it flares up periodically and then clears up for a time.

In one aspect, once an asymptomatic subject is diagnosed as having an allergic disease, treatment can commence immediately to reduce the severity and/or delay the onset of symptoms.

The term “sample” or “patient sample” or “subject sample” or “test sample” can be used generally to refer to a sample of any type which contains products that are to be evaluated by the present methods, including but not limited to, a skin sample including a skin epidermal sample, a skin sample from the Stratum corneum, a tissue sample and/or a bodily fluid sample. The Stratum corneum is the outer layer of the skin (epidermis). It serves as the primary barrier between the body and the environment. The Stratum corneum (SC) is multi layered and is composed of dead, anucleated, flattened corneocytes. The Stratum corneum has a thickness between 10 and 40 μm and can contain about 15-20 layers. In one aspect of the invention, the skin sample comprises skin layers 1, 2, and/or the sum of layers 1 and 2 from the SC. In yet another aspect of the invention, the skin sample comprises skin layers 3, 4, and/or the sum of layers 3 and 4 from the SC. In still another aspect, the skin sample comprises layers 15, 16 and/or the sum of layers 15 and 16 from the SC. In one aspect, the skin sample is taken from non-lesional skin (i.e., skin that appears healthy or normal looking, without any rash). In yet another aspect. The skin sample is taken from lesional skin.

In one aspect, the control sample can be obtained from one or more one or more non-atopic (NA) subjects (subjects that do not have a history of atopic dermatitis).

In one aspect of any of the embodiments related to a method, the subject identified as at risk of developing atopic dermatitis is administered a composition comprising a compound selected from the group consisting of corticosteroids, leukotriene antagonists, anti-cytokine antibodies, anti-cytokine receptor antibodies, anti-IgE antibody, anti-interleukin 14 (IL14) antibodies, anti-interleukin 13 (IL13) antibodies, JAK kinase inhibitors, JAK/STAT inhibitors, antibiotics, a phosphodiesterase inhibitor, a cream comprising filaggrin or components thereof, ceramide rich emollients, and combinations thereof. In one aspect, the composition is administered to the subject by an administration route selected from the group consisting of local administration, topical administration, and injection.

The following examples are provided for illustrative purposes, and are not intended to limit the scope of the invention as claimed herein. Any variations which occur to the skilled artisan are intended to fall within the scope of the present invention.

### EXAMPLES

As provided in the examples below, the findings show that overall, 22/74 (29.7%) and 5/37 (13.5%) infants developed AD in the high risk group and the control group, respectively. In the Stratum corneum (SC) of future AD children, protein-bound ceramides were significantly decreased, and unsaturated sphingomyelin species and “short-chain” NS- and AS-ceramides were elevated as compared to healthy children. Thymic stromal lymphopoietin (TSLP) and interleukin 13 (IL13) levels were increased in the SC of future AD subjects at two months of age (by 74.5% and 78.3%, p=0.0022 and p<0.0001, respectively). Univariable logistic regression analysis revealed strong AD predicting power of the combination of type 2 cytokines and dysregulated lipids, with an odds ratio reaching 51.3.

Method Summary: Newborns (n=111) with and without family history of atopic diseases (risk group, n=74 control group, n=37) were enrolled in Seoul, Korea. Skin tape strips (STS) were collected from the volar area of the forearm at the age of 2 months before any signs of clinical AD, and children were clinically monitored until they reached 2 years of age to ensure the presence or absence of AD. STS were subjected to lipidomic analyses by the LC-MS/MS and cytokine determination by Meso Scale Discovery (MSD) U-Plex assay.

### Example 1

### Methods

### Study Population and Clinical Evaluation

In this birth cohort study, 74 infants in a risk group and 37 infants in a control group were enrolled. At the time of enrollment, parents completed a questionnaire regarding basic demographic information and family history of allergic diseases and underwent SPT with 8 inhalant allergens. On the basis of family history of allergic diseases and SPT response, the risk group was defined when they met one of two criteria: (1) at least one parent had both positive skin test response and history of asthma or allergic rhinitis; and (2) at least one parent or sibling had physician-diagnosed AD. The control group was determined when both parents had neither allergy history nor positive SPT response.

All infants were longitudinally followed for a potential onset of AD until 24 months of age. STS samples were collected at the age of 2 months. Information regarding pregnancy complications, birth date, type of delivery, the child's gestational age, and sex was also obtained. The diagnosis of AD was based on the criteria defined by Hanifin and Rajka (Silverberg N B. Typical and atypical clinical appearance of atopic dermatitis. Clin Dermatol 2017; 35(4): 354-9). At consecutive visits, AD patients were assessed by pediatric allergists using SCORAD score, ranging from 0 to 103 (Chopra R, et al. Severity strata for Eczema Area and Severity Index (EASI), modified EASI, Scoring Atopic Dermatitis (SCORAD), objective SCORAD, Atopic Dermatitis Severity Index and body surface area in adolescents and adults with atopic dermatitis. Br J Dermatol 2017; 177(5): 1316-21); Severity scoring of atopic dermatitis: the SCORAD index. Consensus Report of the European Task Force on Atopic Dermatitis. Dermatology 1993; 186(1): 23-31).

### Participant's Characteristics

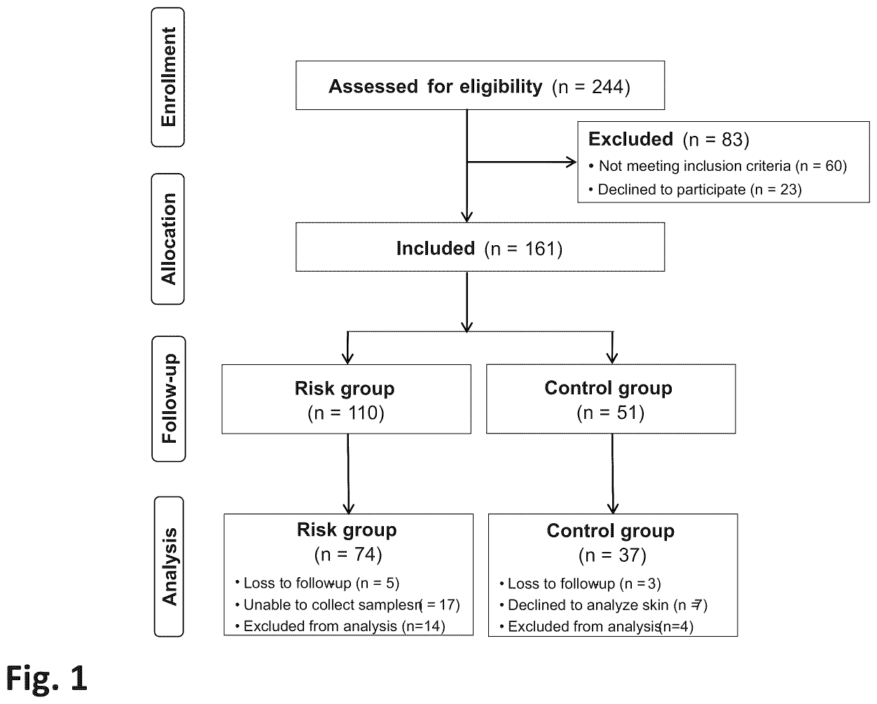

All infants were divided into the control group and risk group for the development of allergic disorders. Family history of allergic diseases and skin prick test (SPT) response when testing parents and siblings were taken into consideration to allocate infants into the control or risk group. Study subjects were assigned to the risk group if they met one of two criteria: (1) at least one parent had both positive skin test response and history of asthma or allergic rhinitis; and (2) at least one parent or sibling had physician-diagnosed AD. Subjects were assigned to the control group if both parents had neither allergy history nor skin test response. Overall, 22/74 (29.7%) and 5/37 (13.5%) infants developed AD in the risk group and the control group, respectively (p=0.060) (FIG. 1A-1B). The mean SCORing Atopic Dermatitis (SCORAD) score in affected infants was 15.7±7.8 (range, 3.7-32.8). The birth cohort was screened for common three filaggrin (FLG) gene mutations, previously reported in Korean population (3321delA, K4022X, and S3296X) (Li K, et al. FLG mutations in the East Asian atopic dermatitis patients: genetic and clinical implication. Exp Dermatol 2016; 25(10): 816-8). Only one infant in the risk group had an FLG gene mutation; this patient had the 3321delA mutation. No babies in the control group had FLG mutations. There were no differences between the risk group and the control group in gender, type of birth (vaginal birth or C-section), preterm birth, use of systemic antibiotics before 6 months of age, introduction of solid foods, family's monthly income, and maternal education levels (Table 2). Children who developed AD together with food allergy were excluded from biochemical analyses as they represent a unique endotype (Leung D Y M, et al. The nonlesional skin surface distinguishes atopic dermatitis with food allergy as a unique endotype. Sci Transl Med 2019; 11(480)). In the univariable logistic regression analysis, there were no significant predictive clinical factors for the development of AD during the first 2 years of life (Table 3).

TABLE 2
Characteristics of study participants (number (%))

| | Risk group | | Control group | | p |
|---|---|---|---|---|---|
| | (n = 79) | | (n = 37) | | value |
| Sex (male) | 39 | (51.9) | 21 | (60.0) | 0.686 |
| Birth type (Cesarean section) | 33 | (44.6) | 12 | (32.4) | 0.219 |
| Preterm birth | 2 | (2.7) | 4 | (10.8) | 0.094 |
| Development of atopic dermatitis | 22 | (29.7) | 5 | (13.5) | 0.060 |
| by the age of 2 Y | | | | | |
| Filaggrin gene mutation | 1 | (1.4) | 0 | (0) | 1.000 |
| Use of systemic antibiotics at | 14 | (18.9) | 3 | (8.1) | 0.169 |
| age <6 mo | | | | | |
| Introduction of solid foods before 6 | 55 | (74.3) | 27 | (73.0) | 0.879 |
| mo | | | | | |
| Monthly income (>4000 US $) | 55 | (74.3) | 25 | (67.6) | 0.454 |
| Maternal education (College) | 64 | (86.5) | 33 | (89.2) | 0.126 |
| Moving to a new house during | 8 | (10.8) | 8 | (21.6) | 0.126 |
| pregnancy | | | | | |
| Mold exposure during pregnancy | 21 | (28.4) | 11 | (29.7) | 0.882 |
| Pet ownership | 7 | (9.5) | 4 | (10.8) | 1.000 |

TABLE 3
Univariable logistic regression analysis of clinical
predictors for development of atopic dermatitis

| Variables | OR (95% CI) | | P value |
|---|---|---|---|
| Sex (male) | 1.08 | (0.45-2.59) | 0.857 |
| Family history of allergic diseases | 2.71 | (0.93-7.86) | 0.067 |
| Birth type (Cesarean section) | 1.50 | (0.60-3.72) | 0.382 |
| Season of birth (winter birth) | 1.24 | (0.51-3.01) | 0.638 |
| Filaggrin gene mutation | | | 1.000 |
| Use of systemic antibiotics at age <6 mo | 2.59 | (0.88-7.66) | 0.086 |
| Introduction of solid foods at age ≥6 mo | 1.26 | (0.48-3.31) | 0.634 |
| Monthly income (>4000 US $) | 1.97 | (0.67-5.78) | 0.216 |
| Maternal education (≥College) | 0.78 | (0.22-2.71) | 0.693 |
| Moving to a new house during pregnancy | 1.51 | (0.47-4.81) | 0.487 |
| Mold exposure during pregnancy | 1.66 | (0.66-4.16) | 0.282 |
| Passive smoking | 0.88 | (0.17-4.1) | 0.878 |

### Skin Prick Tests

Skin prick test (SPT) was performed on the volar aspects of the forearms using the following 8 inhalant allergens in pregnant women and their husbands: Dermatophagoides pteronyssinus, D. farinae, tree pollen mixture I (Alnus glutinosa, Corylus avellana, Populus sp., Ulmus scabra, Salix caprea), tree pollen mixture II (Betula alba, Fagus silvatica, Quercus robur, Platanus orientalis), weed pollen mixture (Artemisia vulgaris, Urtica dioica, Taraxacum vulgare, Plantago lanceolata), grass pollen mixture (Holcus lanatus, Dactylis glomerata, Lolium perenne, Phleum pratense, Poa pratensis, Festuca pratensis, Hordeum vulgare, Avena sativa, Secale cereale, Triticum sativum), cat, and cockroach. Histamine was used as a positive control and normal saline as a negative control. All the above allergens were provided by Allergopharma, Reinbek, Germany. SPT was regarded positive if the wheal diameter was ≥3 mm and controls showed adequate reactions.

### Skin Tape Stripping, Protein Extraction and Mass Spectrometry Analysis

A total of 4 consecutive D-SQUAME® tape strips (22 mm diameter, CuDerm, Dallas, TX, USA) were collected on the volar surface of right forearm at ages of 2 months. On application of the first tape disc, 4 marks were placed around the disc with pen so that subsequent discs could be applied to the same location. Each tape disc was placed adhesive side up in its own 6-well plate and then frozen at −80° C. Strips 5 and 6 were processed for the extraction of free lipids, total sample protein estimation, protein hydrolysis, and re-extraction of protein-bound ceramides as described herein and in Berdyshev, E., et al., Allergy 2022 (Dupilumab significantly improves skin barrier function in patients with moderate-to-severe atopic dermatitis. Allergy. 2022, 77(11):3388-3397). Liquid chromatography tandem mass spectrometry of lipids was performed using a mass spectrometer with an UHPLC front end and an ASCENTIS® Express RP-Amide column (2.7 μm 2.1×50 mm) with gradient elution from methanol:water:formic acid (50:50:0.5, 5 mM ammonium formate) to methanol:chloroform:water:formic acid (90:10:0.5:0.5, 5 mM ammonium formate).

All lipid standards were from AVANTI® Polar Lipids (Birmingham, AL). All ceramide molecules were detected in positive ions mode as a transition from the molecular ion to corresponding sphingoid base minus 2H₂O product ion. Lipid absolute quantitation was achieved using either standard curves of responses of variable amounts of analytes (N-14:0-24:0(C18)S-ceramides versus fixed amount of the internal standard (N-palmitoyl-D-erythro-sphingosine (d7) (d7-ceramide))) or semi quantitatively (EOS-CER, OS-CER, and AS-CER) by comparing lipid signal areas against the signal of corresponding internal standard (N-[26-oleoyloxy(d9) hexacosanoyl]-D-erythro-sphingosine for quantitation of EOS-CER, and N-(2′-(S)-hydroxypalmitoyl(d9)) D-erythro-sphingosine for quantitation of OS-CER and AS-CER). Quantitation of N(C20)S- and N(C22)S-ceramides were achieved using coefficients obtained from the standards curves for N(C18)S-ceramides. Identification of sphingomyelins was performed in positive ions, and quantitation of sphingomyelins was achieved as a transition from the molecular ions to the m/z 184 (phosphocholine) using N-(dodecanoyl)-sphing-4-enine-1-phosphocholine (N12:0-sphingomyelin) as the internal standard and standard curves of variable amounts of different sphingomyelin molecular species (N16:0-N24:0) versus fixed amount of the internal standard.

### Filaggrin Mutation Status

Genomic DNA was extracted from peripheral blood leukocytes and was genotyped for 3 FLG null variants (3321delA, K4022X, and S3296X) which are common among Koreans by direct DNA sequencing as described in Oh H R et al. (Filaggrin Mutation in Korean Patients with Atopic Dermatitis. Yonsei Med J. 2017, 58(2):395-400).

### Human Primary Keratinocyte Culture and Stimulation

Human primary epidermal keratinocytes (HEKs, Thermo Fisher Scientific, Waltham, MA) were grown in serum-free EPILIFETM cell culture medium (Life Technologies, Grand Island, NY) as described in Kim et al., JCI Insight 2021 (Particulate matter causes skin barrier dysfunction. JCI Insight. 2021, 6(5): e145185). To investigate the effects of TSLP on lipoxygenases such as ALOXE3 and ALOXB12, HEKs were differentiated in the presence of 1.3 mmol/L of CaCl₂) for 3 days. Then, cells were incubated with 10 ng/ml of TSLP (R&D Systems, Minneapolis, MN) for 24 hours.

### RNA Preparation and RT-PCR

RNEASY® Mini Kits (QIAGEN®, Valencia, CA) were used according to the manufacturer's protocol to isolate RNA from HEKs. One microgram of RNA was reverse transcribed into cDNA using SUPERSCRIPT® VILO™ MasterMix (Invitrogen, Carlsbad, CA) according to the manufacturer's protocol (Life Technologies). Real-time reverse-transcribed polymerase chain reaction (RT-PCR) was performed and analyzed by the dual-labeled fluorogenic probe method using an ABI Prism 7300 sequence detector (Applied Biosystems, Foster City, CA). Primers and probes for 18s RNA, ALOXE3, and ALOXB12 were purchased from Applied Biosystems. Amplification reactions were performed in MicroAmp optical plates (Applied Biosystems) in a 25-ul volume as described in Kim et al., JCI Insight 2021. Relative expression levels were calculated by the relative standard curve method as outlined in the manufacturer's technical bulletin. Quantities of all targets in test samples were normalized to the corresponding 18s RNA levels in cultured keratinocytes because 18S RNA is very consistently expressed in HEKs stimulated with TSLP.

### Cytokine MSD Assay

Protein extracts were prepared from STS #2, 4 and 8. Tapes were sequentially submerged into eppendorf tubes with PBS. Attached Stratum corneum was removed from the adhesive side of STS by agitation with 5 mm stainless steel beads (QIAGEN®) in TissueLyser (QIAGEN®) for 5 min at 25 Hz. After the procedure, STS were removed from the buffer, protein extracts were centrifuged for 10 min at 14,000 rpm to clear debris and adhesive residue. Samples were then concentrated using Sevant ISS110 Speed Vac Concentrator (ThermoScientific) and cryopreserved for future analysis. Total protein levels in STS extracts were measured with BioradDC protein assay. Protein extracts were analyzed according to the manufacturer's protocol with U-Plex human cytokine multiplex platform on the MESO QuickPlex SQ 120 MM Plate Reader (Meso Scale Discovery, Rockville, MD), which included analytes for human TSLP, IL4 and IL13. Cytokine concentrations were calculated based on standard curves using MSD Workbench Software with customized parameters. For statistical analysis, cytokine concentrations below the fit curve range (signal below the bottom of the bottom-of-the-curve fit, no concentration given) were extrapolated below the standard curve detection limit to maintain the ranking order. The MSD assay results for each measured cytokine were normalized to the total protein amount in each sample.

### Statistical Analysis

Data were analyzed using SPSS® for Windows (version 27.0, SPSS, Chicago, USA) and GraphPad Prism (version 9.3.0, GraphPad Software, San Diego, CA, USA). The Chi-squared test and Fisher exact test were applied to determine the differences in the proportions. Shapiro-Wilk test was used to determine whether data was distributed normally. Epidermal lipid profile levels were compared using Mann-Whitney U test between infants with AD and those without AD.

Univariable logistic regression analysis was conducted to determine the effect of cytokines and lipids on the development of AD. Variables for adjustment included sex, family history of allergic diseases, type of delivery, FLG mutation, birth season, maternal education levels, antibiotic treatment during the first 6 months of life, the age of introduction of solid foods, passive smoking, moving to a new house during pregnancy, and exposure to mold during pregnancy.

The cutoff levels for cytokines and lipids at the age of 2 months were determined by analyzing the receiver operating characteristic (ROC) curve. ROC curves were analyzed and area under curves (AUC) were measured to estimate the diagnostic values of each analyte to predict the development of AD. The infants were divided into two groups according to these cutoff levels at the age of 2 months: high levels vs. low levels. The combined effect of cytokines, lipids, and family history was also evaluated using a univariable logistic regression. A P value <0.05 was considered to be significant.

### Example 2

### TSLP and IL13 Levels are Increased in STS of 2-Month-Old Future Clinical AD Patients

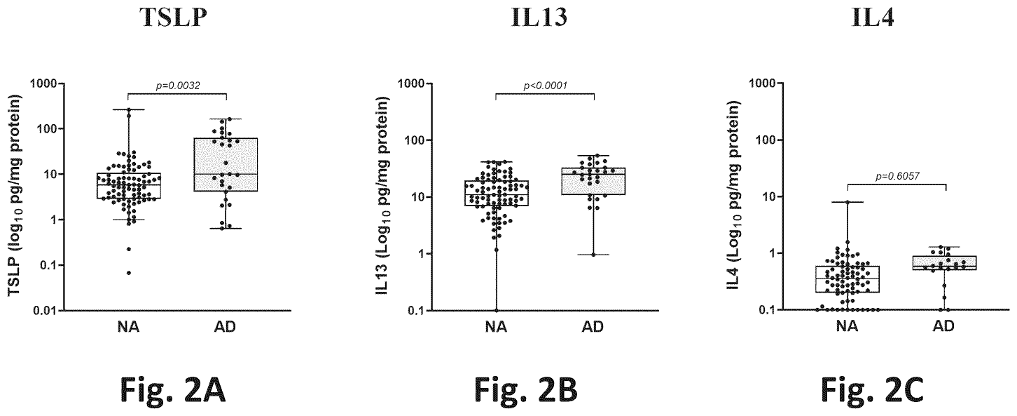

Previously TSLP was identified in the SC at the age of 2 months as a predictor of future clinical AD (Kim J, et al. Epidermal thymic stromal lymphopoietin predicts the development of atopic dermatitis during infancy. J Allergy Clin Immunol 2016; 137(4): 1282-5 e4). To confirm this observation in a separate independent cohort, TSLP levels in STS protein extracts from the current replication cohort was analyzed. As shown in FIG. 2A, at the age of 2 months in healthy appearing infants, TSLP levels in STS of future AD subjects was statistically significantly higher in comparison to its level in future healthy children STS samples. As TSLP is known to induce type 2 cytokines IL4 and IL13 in lymphocytes that are considered the main drivers of AD, (Leung D Y M, et al. Cutaneous barrier dysfunction in allergic diseases. J Allergy Clin Immunol 2020; 145(6): 1485-97SC) protein preparations was also tested for their levels. As shown in FIGS. 2B and 2C, IL13 levels were also elevated in STS from future AD subjects, while the levels of IL4 was not substantially changed.

### Example 3

Lipid Profiles in Stratum corneum (Protein-Bound OS-CER, EOS-CER, NS-CER, AS-CER, Sphingomyelins) are Altered in Infants with Future Clinical AD

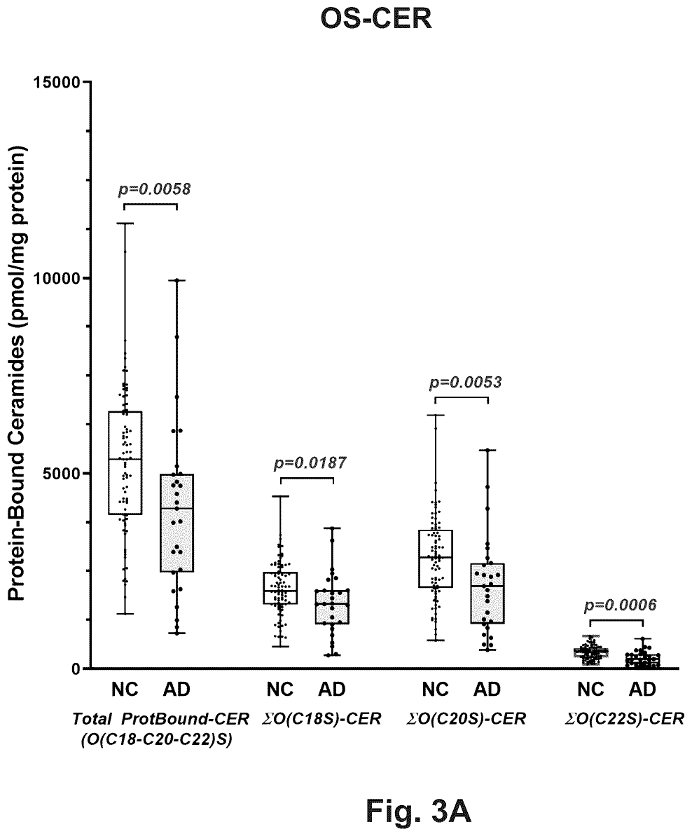

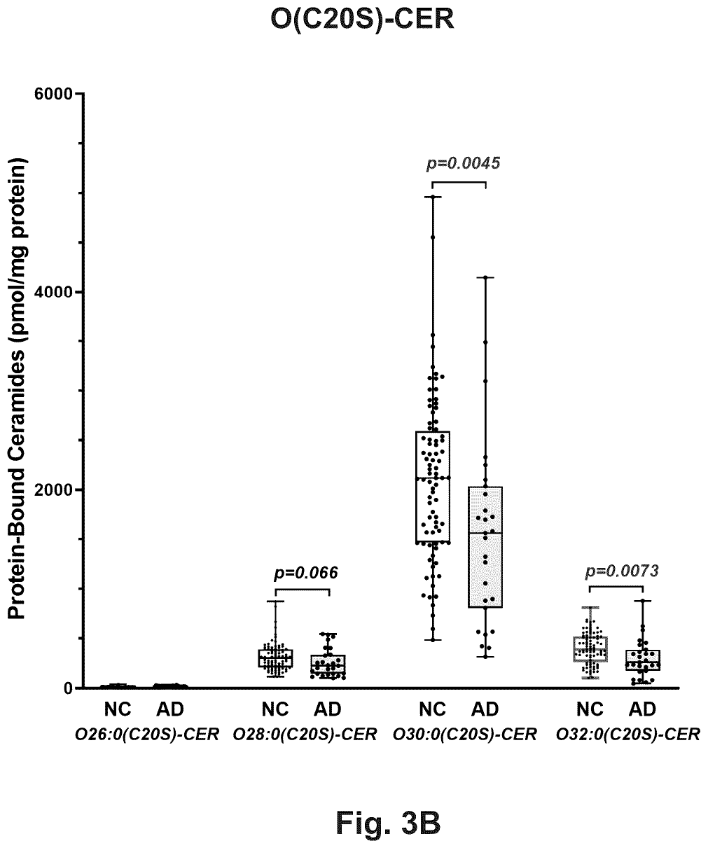

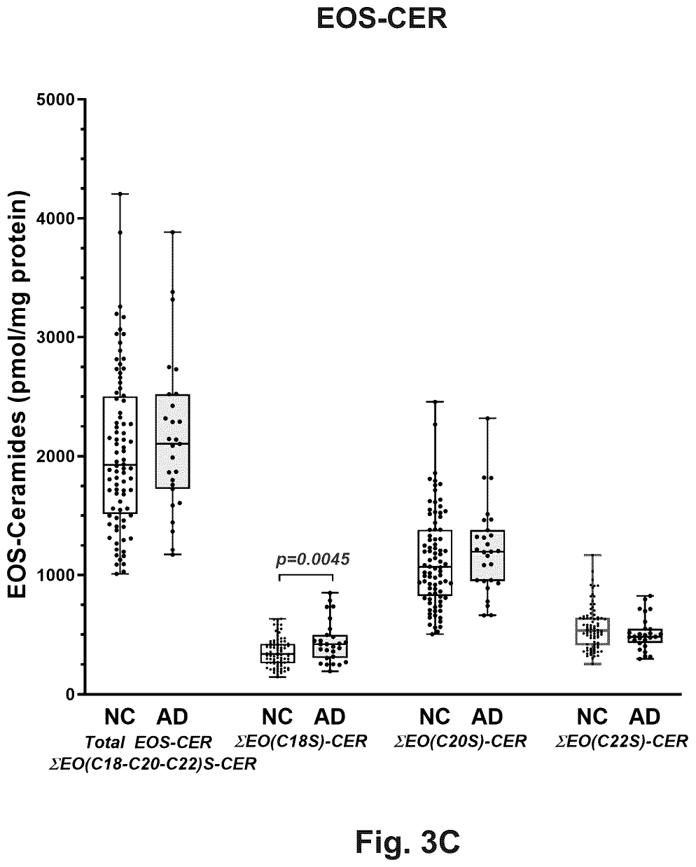

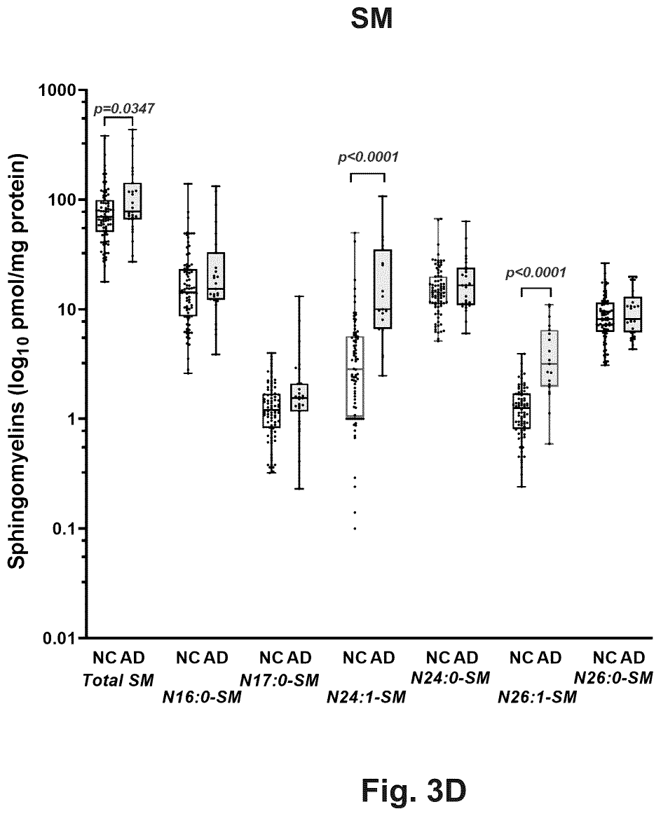

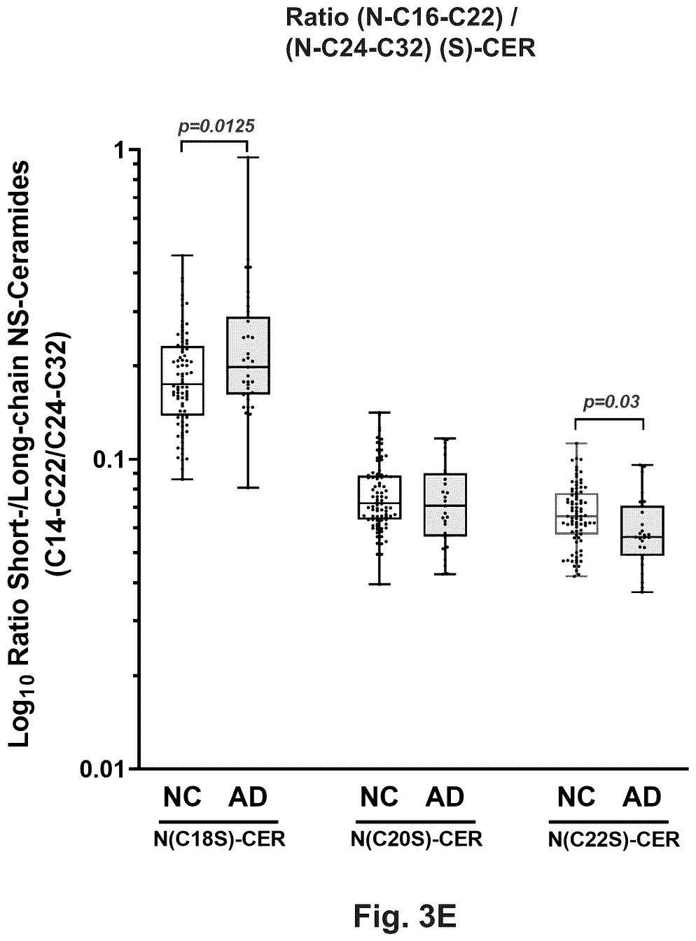

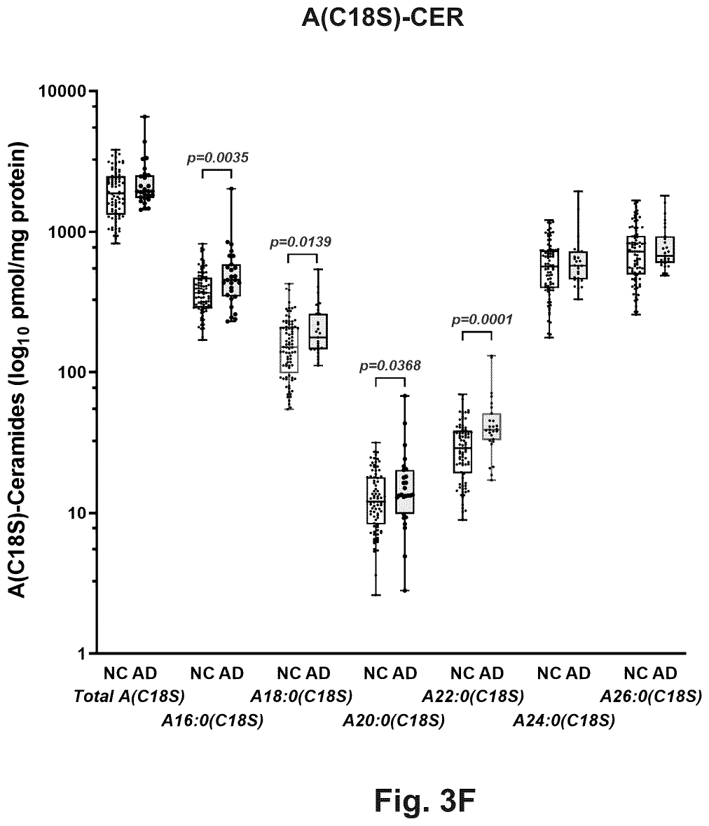

To find novel predictive biomarkers of future AD, STS layers 5,6 from the volar area of the forearm of 2-month-old infants was analyzed for the panel of SC lipids including protein-bound OS-ceramides (OS-CER). At the age of 2 months, OS-CER were significantly decreased in the SC of infants who developed AD within 2 years after birth. This decrease was seen in all three groups of analyzed OS-ceramides (with C18-, C20-, and C22-sphingosine as a sphingoid base) and within all analyzed main molecular species (FIG. 3A, FIG. 3B). The levels of the immediate precursors of OS-CER, the esterified OS-CER (EOS-CER), were not modified in the SC at the age of 2 months with the exception of EO(C18S)-ceramides, which level was slightly increased (FIG. 3C).

Increased levels of sphingomyelins and non-hydroxy fatty acid sphingosine ((NS)-ceramides with C18-sphingosine (N(C18S)-CER) are known to be associated with developed AD (Berdyshev E, et al. Lipid abnormalities in atopic skin are driven by type 2 cytokines. JCI Insight 2018; 3(4); Berdyshev E, et al. Signaling sphingolipids are biomarkers for atopic dermatitis prone to disseminated viral infections. J Allergy Clin Immunol 2022; 150(3): 640-8). At the age of 2 months, several months before the onset of AD, the levels of two prominent unsaturated sphingomyelin molecular species (24:1- and 26:1-SM) were significantly upregulated in the SC of future AD subjects, enough to ensure a significant upregulation of sphingomyelin total level, regardless that the saturated sphingomyelin molecular species were not different between healthy and future AD groups (FIG. 3D). Among individual molecular species of non-hydroxy fatty acid sphingosine-containing ceramides (NS-CER), only “short-chain” N16:0(C18S)-ceramide demonstrated minimal upregulation in the future AD group versus healthy group (with averages as 150 vs 81 pmol/mg protein, correspondingly, p=0.0003). Also, looking at NS-CER globally, the ratio between “short-chain” fatty acid (C14-C22) and “long-chain” fatty acid (C24-C32) containing NS-CER with C18-sphingosine was significantly upregulated, while the same ratio in C22-sphingosine-containing ceramides was decreased in future AD subjects (FIG. 3E). No changes were observed in C20-sphingosine containing NS-CER (FIG. 3E).

Similar to NS-CER, alpha-hydroxy fatty acid containing ceramides (AS-CER) with C18-sphingosine also demonstrated the difference between healthy and future AD groups. Thus, multiple “short-chain” AS-CER with C18-sphingosine molecular species were upregulated in future AD subjects, with A16:0(C18S)-ceramide being the most abundant upregulated AS-CER molecular specie (FIG. 3F). AS-CER with C20- and C22-sphingosine did not demonstrate substantial differences between AD and NA groups (Table 1).

### Example 4

FLG Breakdown Products were not Predictors of the Future AD Onset

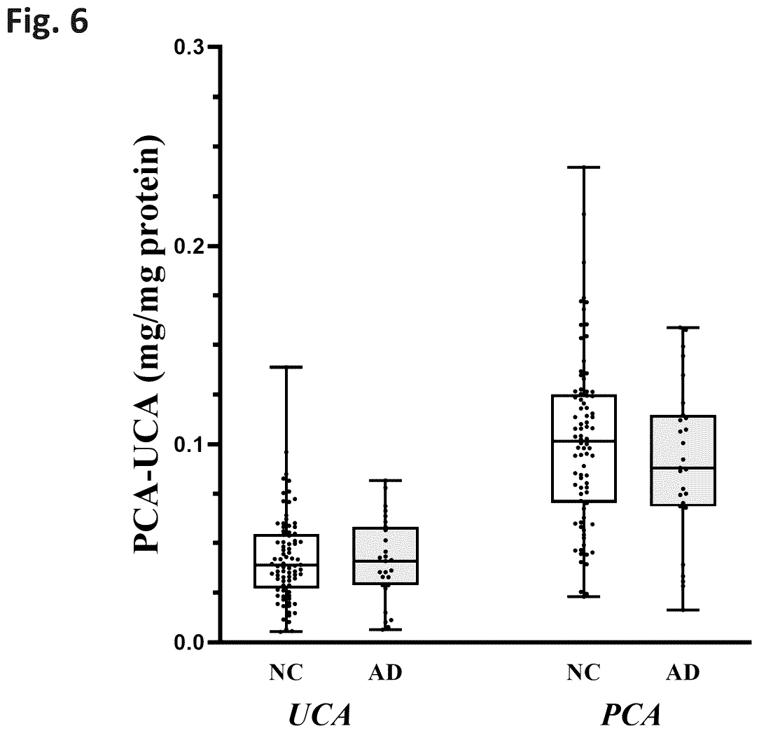

Clinical AD in adults and adolescents is strongly associated with decreased expression of FLG protein and decreased levels of FLG breakdown products urocanic acid (UCA) and pyroglutamic acid (PCA) (Drislane C, Irvine A D. The role of filaggrin in atopic dermatitis and allergic disease. Ann Allergy Asthma Immunol 2020; 124(1): 36-43). To check if FLG breakdown product levels are predictive for the future AD onset, polar components of SC for the levels of UCA and PCA were analyzed. As shown in FIG. 6, no difference was observed between healthy and future AD subjects.

### Example 5

### TSLP Inhibits Expression of ALOXE3 and ALOX12B in Primary Human Keratinocytes In Vitro

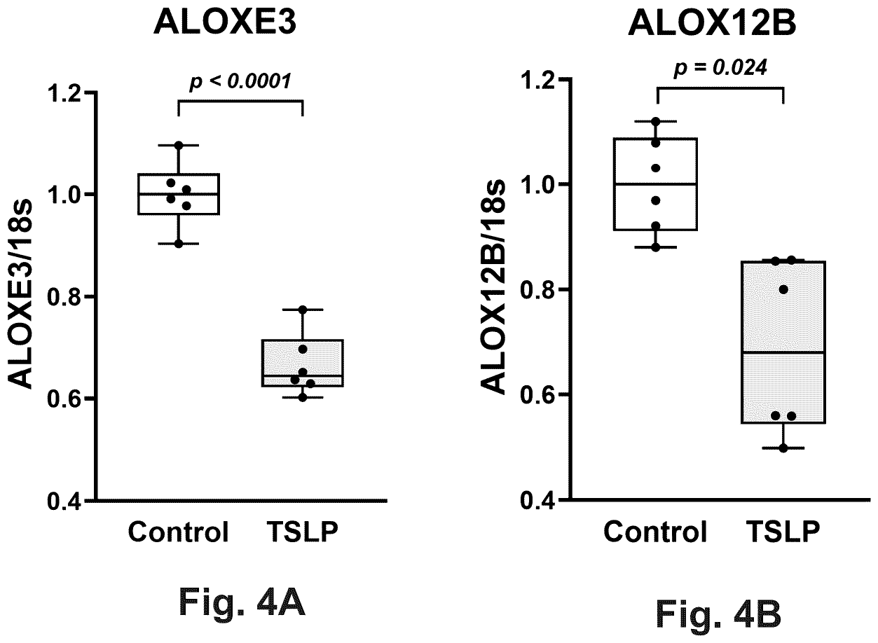

As protein-bound ceramides were found to be decreased in STS of future clinical AD (FIG. 3A, FIG. 3B), and TSLP was shown previously (Kim J, et al. Epidermal thymic stromal lymphopoietin predicts the development of atopic dermatitis during infancy. J Allergy Clin Immunol 2016; 137(4): 1282-5 e4) and in this cohort (FIGS. 2A-2C) to be elevated in the SC of future AD subjects at the age of 2 months, TSLP was further examined to determine whether it regulates the expression of EOS-CER processing enzymes. To this end, Ca²⁺-differentiated human epidermal keratinocytes (HEKs) were treated in vitro with TSLP (10 ng/mL) for 24 h, and the expressions of ALOXE3 and ALOX12B enzymes were evaluated by reverse transcription-polymerase chain reaction (RT-PCR). As shown in the FIGS. 4A and 4B, TSLP strongly inhibited the transcription of ALOXE3 (p<0.0001) and ALOX12B (p<0.05) genes.

### Example 6

### Powerful Biomarkers to Predict AD Development: Combination of Lipids and Cytokines

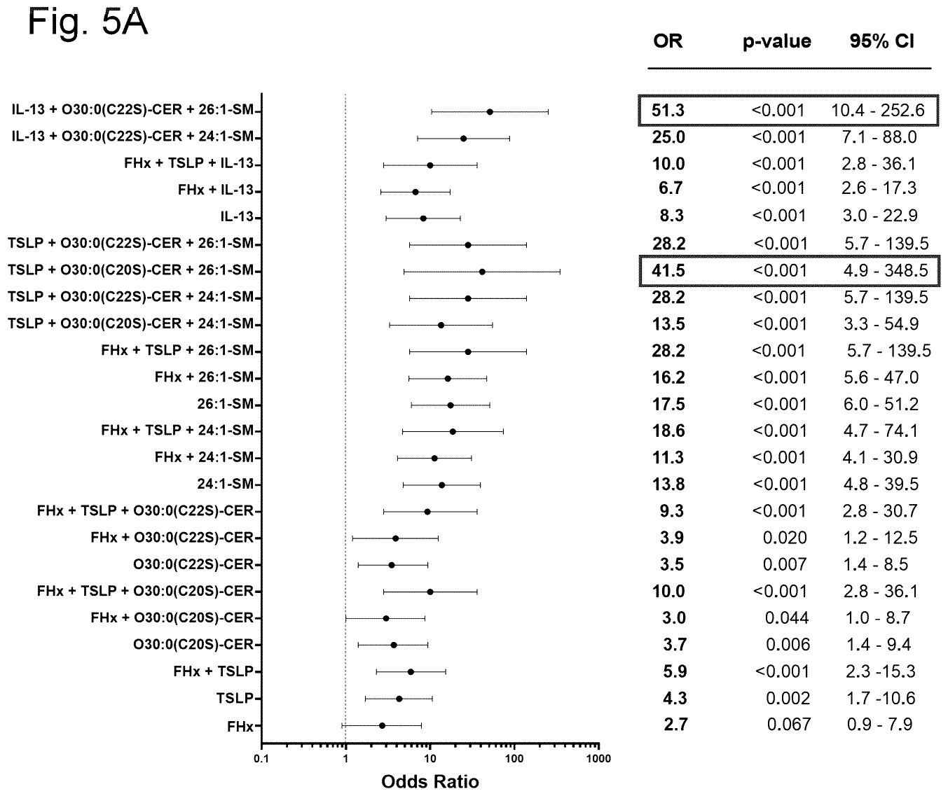

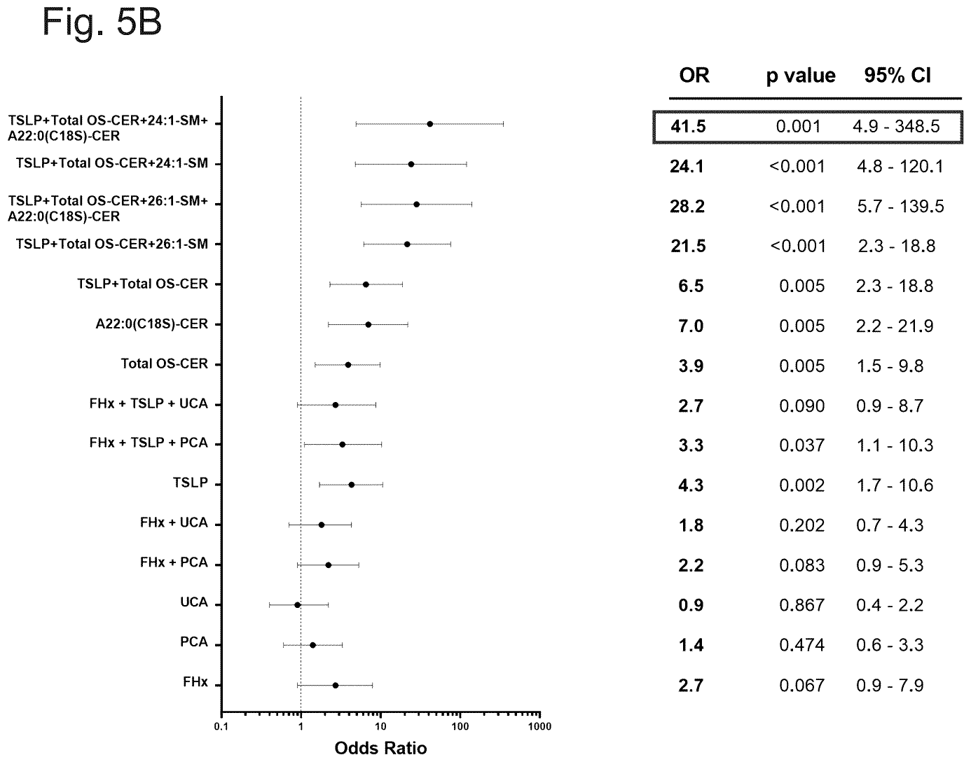

In this new cohort, first, a cutoff value for cytokines and lipids were determined by analyzing the receiver operating characteristic (ROC) curves using data obtained from STS collected at 2 months of age (Table 4). A univariable logistic regression analysis revealed that STS level of TSLP at the age of 2 months is again (as previously shown) (Kim J, et al. Epidermal thymic stromal lymphopoietin predicts the development of atopic dermatitis during infancy. J Allergy Clin Immunol 2016; 137(4): 1282-5 e4) predictive of the onset of AD by the age of 24 months with the OR of 4.3 (95% CI 1.7-10.6). When combined with family history of atopic diseases, the predictive power of STS TSLP level rose to the OR of 5.9 (with 95% CI of 2.3-15.3) (FIGS. 5A and 5B).

TABLE 4
Cutoff values determined using ROC data assessment for
parameters that predict the onset of AD at the age of
24 months when STS are analyzed at the age of 2 months

| Variables | Cutoff value | |
|---|---|---|
| Family history (FHx) | yes | |
| TSLP | ≥8.2 | pg/mg protein |
| IL13 | ≥16.7 | pg/mg protein |
| UCA | <0.039 | mg/mg protein |
| PCA | <0.1 | mg/mg protein |
| O30:0(C20S)-CER | <1828.0 | pmol/mg protein |
| O30:0(C22S)-CER | <366.0 | pmol/mg protein |
| Total OS-CER | <4824.2 | pmol/mg protein |
| 24:1-SM | ≥6.4 | pmol/mg/protein |
| 26:1-SM | ≥1.9 | pmol/mg/protein |
| A22:0(C18S)-CER | ≥30.7 | pmol/mg/protein |

Protein-bound OS-CER are critical for the proper assembly of the cornified envelope (Elias P M, et al. Formation and functions of the corneocyte lipid envelope (CLE). Biochim Biophys Acta 2014; 1841(3): 314-8). The dichotomization of OS-CER levels in the SC at the age of 2 months indicated that the level of the most abundant O30:0(C20S)-CER molecular species below 1828 pmol/mg protein separates subjects at risk of future AD development and healthy subjects (Table 4). Univariable logistic regression analysis revealed that low levels of individual OS-CER species as well as low level of total OS-CER increase the OR of future onset of AD to 3.5-3.9, and accounting for a family history of atopic diseases did not add to the predictive power of OS-CER species (FIGS. 5A and 5B). However, the combination of family history, low TSLP, and low O30:0(C20S)-CER increased the OR of future onset of AD to 10 (95% CI 2.8-36.1) (FIGS. 5A and 5B).

Interestingly, monounsaturated molecular species of sphingomyelin 24:1-SM and 26:1-SM were determined to be even better predictors of future AD onset than OS-ceramides. Their content was elevated in the SC of future AD subjects (FIGS. 3A-3F), and high levels of 24:1-SM and 26:1-SM have increased the chance of future AD onset, with ORs of 13.8 (95% CI 4.8-39.5) and 17.5 (95% CI 6.0-51.2), respectively (FIGS. 5A and 5B). Moreover, the combined presence of high levels of monounsaturated SMs, TSLP, and the family history of atopic diseases increased the ORs to 18.6 (24:1-SM, 95% CI 4.7-74.1) and 28.2 (26:1-SM, 95% CI 5.7-139.5). However, one of the highest OR of developing AD by the age of 24 months was obtained when accounting for family history, high TSLP, high 26:1-SM, and low O30:0(C20S)-CER (OR=41.5, 95% CI 4.9-348.5). These data point out the high power of combinatorial assessment of biomarkers of different etiology to predict future onset of AD, with novel lipid biomarkers contributing the most to this assessment.

IL4 and IL13 cytokines are TSLP-dependent cytokines (Ziegler S F. Thymic stromal lymphopoietin, skin barrier dysfunction, and the atopic march. Ann Allergy Asthma Immunol 2021; 127(3): 306-11). At the age of 2 months, in addition to TSLP, IL13 was found to be significantly increased in the SC of future AD subjects (FIG. 2B). According to univariable logistic analysis, the increased IL13 levels were determined to be even better predicting parameter of the AD onset by the age of 24 months than TSLP (FIGS. 5A and 5B). Thus, high IL13 levels alone predicted future onset of AD with OR of 8.3 (95% CI 3.0-22.9). Interestingly, accounting for a family history of atopic disease alone or in combination with high levels of TSLP did not further increase the OR of future onset of AD (FIGS. 5A and 5B).

The AS-ceramide, A22:0(C18S)-CER, was also found as a novel good predictor of the future onset of AD by the age of 24 months. Alone, high levels of A22:0(C18S)-CER signified the OR of 7.0 (95% CI 2.2-21.9) of the future of AD development (FIGS. 5A and 5B), and in combination with high TSLP, high 24:1-SM, and low total OS-ceramides, the OR rose to 41.5 (95% CI 4.9-348.5). Importantly, when combined with high IL13, high 26:1-SM, and low O30:0(C22S)-CER levels, low A22:0(C18S)-CER level predicted the future onset of AD with OR of 51.3 (95% CI 10.4-252.6). Thus, these findings not only confirmed TSLP as a good predictor of the AD onset by the age of 24 months but also identified IL13, protein-bound ceramides, unsaturated sphingomyelins, and AS-ceramides as novel strong STS biomarkers at the age of 2 months for the future development of AD.

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### REFERENCES

1. Park Y M, Lee S Y, Kim W K, et al. Risk factors of atopic dermatitis in Korean schoolchildren: 2010 international study of asthma and allergies in childhood. Asian Pac J Allergy Immunol 2016; 34(1): 65-72.
2. Abuabara K, Yu A M, Okhovat J P, Allen I E, Langan S M. The prevalence of atopic dermatitis beyond childhood: A systematic review and meta-analysis of longitudinal studies. Allergy 2018; 73(3): 696-704.
3 Kim J, Kim B E, Leung D Y M. Pathophysiology of atopic dermatitis: Clinical implications. Allergy Asthma Proc 2019; 40(2): 84-92.
4. Paller A S, Spergel J M, Mina-Osorio P, Irvine A D. The atopic march and atopic multimorbidity: Many trajectories, many pathways. J Allergy Clin Immunol 2019; 143(1): 46-55.
5. Leung D Y M, Berdyshev E, Goleva E. Cutaneous barrier dysfunction in allergic diseases. J Allergy Clin Immunol 2020; 145(6): 1485-97.
6. Luger T, Amagai M, Dreno B, et al. Atopic dermatitis: Role of the skin barrier, environment, microbiome, and therapeutic agents. J Dermatol Sci 2021; 102(3): 142-57.
7. Skjerven H O, Rehbinder E M, Vettukattil R, et al. Skin emollient and early complementary feeding to prevent infant atopic dermatitis (PreventADALL): a factorial, multicentre, cluster-randomised trial. Lancet 2020; 395(10228): 951-61.
8 Bradshaw L E, Wyatt L A, Brown S J, et al. Emollients for prevention of atopic dermatitis: 5-year findings from the BEEP randomized trial. Allergy 2022.
9. Zhong Y, Samuel M, van Bever H, Tham E H. Emollients in infancy to prevent atopic dermatitis: A systematic review and meta-analysis. Allergy 2022; 77(6): 1685-99.
10. Kim J, Kim B E, Lee J, et al. Epidermal thymic stromal lymphopoietin predicts the development of atopic dermatitis during infancy. J Allergy Clin Immunol 2016; 137(4): 1282-5 e4.
11. Li K, Oh W J, Park K Y, Kim K H, Seo S J. FLG mutations in the East Asian atopic dermatitis patients: genetic and clinical implication. Exp Dermatol 2016; 25(10): 816-8.
12. Leung D Y M, Calatroni A, Zaramela L S, et al. The nonlesional skin surface distinguishes atopic dermatitis with food allergy as a unique endotype. Sci Transl Med 2019; 11(480).
13. Berdyshev E, Goleva E, Bronova I, et al. Lipid abnormalities in atopic skin are driven by type 2 cytokines. JCI Insight 2018; 3(4).
14. Berdyshev E, Goleva E, Bronova I, et al. Signaling sphingolipids are biomarkers for atopic dermatitis prone to disseminated viral infections. J Allergy Clin Immunol 2022; 150(3): 640-8.
15. Drislane C, Irvine A D. The role of filaggrin in atopic dermatitis and allergic disease. Ann Allergy Asthma Immunol 2020; 124(1): 36-43.
16. Elias P M, Gruber R, Crumrine D, et al. Formation and functions of the corneocyte lipid envelope (CLE). Biochim Biophys Acta 2014; 1841(3): 314-8.
17. Ziegler S F. Thymic stromal lymphopoietin, skin barrier dysfunction, and the atopic march. Ann Allergy Asthma Immunol 2021; 127(3): 306-11.
18. Nemes Z, Marekov L N, Fesus L, Steinert P M. A novel function for transglutaminase 1: attachment of long-chain omega-hydroxyceramides to involucrin by ester bond formation. Proc Natl Acad Sci USA 1999; 96(15): 8402-7.
19. Marekov L N, Steinert P M. Ceramides are bound to structural proteins of the human foreskin epidermal cornified cell envelope. J Biol Chem 1998; 273(28): 17763-70.
20. Rinnov M R, Halling A S, Gerner T, et al. Skin biomarkers predict development of atopic dermatitis in infancy. Allergy 2022.
21. Nishifuji K, Yoon J S. The Stratum corneum: the rampart of the mammalian body. Vet Dermatol 2013; 24(1): 60-72 e15-6.
22. Candi E, Schmidt R, Melino G. The cornified envelope: a model of cell death in the skin. Nat Rev Mol Cell Biol 2005; 6(4): 328-40.
23. Howell M D, Kim B E, Gao P, et al. Cytokine modulation of atopic dermatitis filaggrin skin expression. J Allergy Clin Immunol 2007; 120(1): 150-5.
24. Irvine A D, McLean W H, Leung D Y. Filaggrin mutations associated with skin and allergic diseases. N Engl J Med 2011; 365(14): 1315-27.
25. Kurihara M, Kabata H, Irie M, Fukunaga K. Current summary of clinical studies on anti-TSLP antibody, Tezepelumab, in asthma. Allergol Int 2022.
26. Koskeridis F, Evangelou E, Ntzani E E, Kostikas K, Tsabouri S. Treatment With Dupilumab in Patients With Atopic Dermatitis: Systematic Review and Meta-Analysis. J Cutan Med Surg 2022; 26(6): 613-21.
27. Kelly K A, Perche P O, Feldman S R. Therapeutic Potential of Tralokinumab in the Treatment of Atopic Dermatitis: A Review on the Emerging Clinical Data. Clin Cosmet Investig Dermatol 2022; 15: 1037-43.
28. Silverberg N B. Typical and atypical clinical appearance of atopic dermatitis. Clin Dermatol 2017; 35(4): 354-9.
29. Chopra R, Vakharia P P, Sacotte R, et al. Severity strata for Eczema Area and Severity Index (EASI), modified EASI, Scoring Atopic Dermatitis (SCORAD), objective SCORAD, Atopic Dermatitis Severity Index and body surface area in adolescents and adults with atopic dermatitis. Br J Dermatol 2017; 177(5): 1316-21.
30. Severity scoring of atopic dermatitis: the SCORAD index. Consensus Report of the European Task Force on Atopic Dermatitis. Dermatology 1993; 186(1): 23-31.
31. Berdyshev E, Goleva E, Bissonnette R, Bronova I, Bronoff A S, Richers B N, Garcia S, Ramirez-Gama M, Taylor P, Praestgaard A, Agueusop I, Jurvilliers P, Boguniewicz M, Levit N A, Rossi A B, Zhang A, Leung D Y M. Dupilumab significantly improves skin barrier function in patients with moderate-to-severe atopic dermatitis. Allergy. 2022, 77(11):3388-3397.
32. On H R, Lee S E, Kim S E, Hong W J, Kim H J, Nomura T, Suzuki S, Shimizu H, Kim S C. Filaggrin Mutation in Korean Patients with Atopic Dermatitis. Yonsei Med J. 2017, 58(2):395-400.
33. Kim B E, Kim J, Goleva E, Berdyshev E, Lee J, Vang K A, Lee U H, Han S, Leung S, Hall C F, Kim N R, Bronova I, Lee E J, Yang H R, Leung D Y, Ahn K. Particulate matter causes skin barrier dysfunction. JCI Insight. 2021, 6(5):e145185.

What is claimed is:

1. A method to identify and/or screen a human infant at risk of developing atopic dermatitis (AD), wherein the infant has not been diagnosed as having AD and/or an atopic disease, the method comprising:
a. obtaining a skin sample from the infant, wherein the skin sample is a non-lesional skin sample from the infant;
b. determining from the skin sample an expression level of a cytokine and/or lipid selected from the group consisting of interleukin 13 (IL13), thymic stromal lymphopoietin (TSLP), one or more ceramides, one or more sphingomyelins, and a combination thereof; and
c. comparing the expression level of IL13, TSLP, one or more ceramides, one or more sphingomyelins, or a combination thereof, in the skin sample to a control sample wherein the control sample is from one or more non-atopic (NA) subjects;
wherein an elevated level of IL13 and/or TSLP as compared to the control sample identifies the infant as being at risk of developing AD; and/or
wherein a decrease in one or more ceramides as compared to the control sample identifies the infant as being at risk of developing AD; and/or
wherein an increase in one or more sphingomyelins as compared to the control sample identifies the infant as being at risk of developing AD.

2. The method of claim 1, wherein the ceramides are protein bound ceramides.

3. The method of claim 2, wherein the protein bound ceramides are ceramides comprising alpha hydroxy fatty acids.

4. The method of claim 1, wherein the sphingomyelins are sphingomyelins comprising unsaturated fatty acids.

5. The method of claim 1, wherein the skin sample is obtained by a skin tape stripping method.

6. The method of claim 5, wherein the skin tape stripping method comprises:
a. applying an adhesive tape to a target area of the skin of the infant in a manner sufficient to isolate an epidermal sample adhering to the adhesive tape, wherein the epidermal sample comprises cells from the Stratum corneum of the infant, wherein the tape comprises a rubber adhesive;
b. extracting the epidermal sample comprising the cells adhering to the adhesive tape with a cell scraper comprising thermoplastic elastomer material in a solvent of about 5% to about 30% alcohol in water; and
c. determining in the extracted epidermal sample an expression level of IL13, TSLP, one or more ceramides, one or more sphingomyelins, or a combination thereof.

7. A method of preemptive intervention in a human infant at risk of developing AD, wherein the infant has not been diagnosed as having AD and/or an atopic disease, the method comprising:
a. obtaining a skin sample from the infant, wherein the skin sample is a non-lesional skin sample from the infant;
b. determining from the skin sample an expression level of a cytokine and/or lipid selected from the group consisting of IL13, TSLP, one or more ceramides, one or more sphingomyelins, and a combination thereof; and
c. comparing the expression level of IL13, TSLP, one or more ceramides, one or more sphingomyelins, or a combination thereof, in the skin sample to a control sample wherein the control sample is from one or more non-atopic (NA) subjects;
wherein an elevated level of IL13 and/or TSLP as compared to the control sample identifies the infant as being at risk of developing AD; and/or
wherein a decrease in one or more ceramides as compared to the control sample identifies the infant as being at risk of developing AD; and/or
wherein an increase in one or more sphingomyelins as compared to the control sample identifies the infant as being at risk of developing AD; and
d. providing intervention to the infant identified as being at risk of developing AD, wherein the intervention is selected from the group consisting of administering an immune modifier, a skin barrier enforcing emollient and combinations thereof to the infant.

8. The method of claim 7, wherein the ceramides are protein bound ceramides.

9. The method of claim 8, wherein the protein bound ceramides are ceramides comprising alpha hydroxy fatty acids.

10. The method of claim 7, wherein the sphingomyelins are sphingomyelins comprising unsaturated fatty acids.

11. The method of claim 7, wherein the skin sample is obtained by a skin tape stripping method.

12. The method of claim 11, wherein the skin tape stripping method comprises:
a. applying an adhesive tape to a target area of the skin of the infant in a manner sufficient to isolate an epidermal sample adhering to the adhesive tape, wherein the epidermal sample comprises cells from the Stratum corneum of the infant, wherein the tape comprises a rubber adhesive;
b. extracting the epidermal sample comprising the cells adhering to the adhesive tape with a cell scraper comprising thermoplastic elastomer material in a solvent of about 5% to about 30% alcohol in water; and
c. determining in the extracted epidermal sample an expression level of IL13, TSLP, one or more ceramides, one or more sphingomyelins, or a combination thereof.

13. The method of claim 7, wherein the immune modifier is selected from the group consisting of an anti-TSLP biologic, an anti-IL4/IL13 biologic and combinations thereof.

14. A method to prevent and/or decrease the severity of an atopic disease in a human infant at risk of developing AD, wherein the infant has not been diagnosed as having AD and/or an atopic disease, the method comprising:
a. obtaining a skin sample from the infant, wherein the skin sample is a non-lesional skin sample from the infant;
b. determining from the skin sample an expression level of a cytokine and/or lipid selected from the group consisting of IL13, TSLP, one or more ceramides, one or more sphingomyelins, and a combination thereof; and
c. comparing the expression level of IL13, TSLP, one or more protein-bound ceramides, one or more sphingomyelins, or a combination thereof, in the skin sample to a control sample wherein the control sample is from one or more non-atopic (NA) subjects;
wherein an elevated level of IL13 and/or TSLP as compared to the control sample identifies the infant as being at risk of developing AD; and/or
wherein a decrease in one or more ceramides as compared to the control sample identifies the infant as being at risk of developing AD; and/or
wherein an increase in one or more sphingomyelins as compared to the control sample identifies the infant as being at risk of developing AD; and
d. administering to the infant identified as being at risk of developing AD, an immune modifier, a skin barrier enforcing emollient and combinations thereof to the infant.

15. The method of claim 14, wherein the ceramides are protein bound ceramides.

16. The method of claim 15, wherein the protein bound ceramides are ceramides comprising alpha hydroxy fatty acids.

17. The method of claim 14, wherein the sphingomyelins are sphingomyelins comprising unsaturated fatty acids.

18. The method of 14, wherein the skin sample is obtained by a skin tape stripping method.

19. The method of claim 18, wherein the skin tape stripping method comprises:
a. applying an adhesive tape to a target area of the skin of the infant in a manner sufficient to isolate an epidermal sample adhering to the adhesive tape, wherein the epidermal sample comprises cells from the Stratum corneum of the infant, wherein the tape comprises a rubber adhesive;
b. extracting the epidermal sample comprising the cells adhering to the adhesive tape with a cell scraper comprising thermoplastic elastomer material in a solvent of about 5% to about 30% alcohol in water; and
c. determining in the extracted epidermal sample an expression level of IL13, TSLP, one or more ceramides, one or more sphingomyelins, or a combination thereof.

20. The method of claim 14, wherein the immune modifier is selected from the group consisting of an anti-TSLP biologic, an anti-IL4/IL13 biologic and combinations thereof.
